(19) United States
(12) Patent Application Publication
(10) Pub. No.: US 2020/0318166 A1
(43) Pub. Date: Oct. 8, 2020

(54) Multiplexed Screening

(71) Applicant: Altius Institute for Biomedical Sciences, Seattle, WA (US)

(72) Inventors: John A. Stamatoyannopoulos, Seattle, WA (US); Shreeram Akilesh, Seattle, WA (US); Pavel Zrazhevskiy, Seattle, WA (US)

(21) Appl. No.: 16/478,448

(22) PCT Filed: Jan. 18, 2018
(86) PCT No.: PCT/US2018/014292

§ 371 Date: Jul. 16, 2019

Related U.S. Application Data
(60) Provisional application No. 62/447,793, filed on Jan. 18, 2017.

(51) Int. Cl. C12Q 1/6816

(52) U.S. Cl. CPC C12Q 1/6816; C12Q 2537/143; C12Q 2521/301

(57) ABSTRACT

Methods and apparatus to test and screen compounds in a multiplexed manner, using a mixture of genetically or functionally heterogeneous cells in common conditions.

### CROSS-REFERENCE

This application claims the benefit of U.S. Provisional Patent Application No. 62/447,793, filed Jan. 18, 2017, the entire disclosure of which is incorporated by reference herein.

### BACKGROUND

A chemical library or compound library is a collection of stored chemicals or compounds. These chemicals or compounds have the potential to treat a wide variety of diseases or disorders. However, the utility of these chemicals or compounds is constrained by the ability to test each chemical or compound against a given disease or disorder. Therefore, methods and apparatuses for effectively and efficiently screening a large number of chemicals or compounds against a variety of disease-relevant cell types are needed.

### SUMMARY

Described herein are methods of multiplexed screening, the methods comprising: providing a plurality of vessels, wherein each vessel comprises: a first biological cell comprising a first detectable marker and a first genotype and a second biological cell comprising a second detectable marker and a second genotype, wherein the second genotype comprises a genetic variation relative to the first genotype; contacting the first biological cell and the second biological cell with a compound in each vessel; and detecting the first detectable marker and the second detectable marker after the contacting in each vessel. In some instances, the methods further comprise quantifying the level of the first detectable marker and the second detectable marker in each vessel. In some instances, the methods further comprise analyzing the first biological cell or second biological cell using flow cytometry. In some instances, the detecting is by mass spectrometry, optical detection, or microscopy.

In some instances, the first detectable marker is a fluorescent marker or an isotopic label, and in some instances, the first detectable marker labels a membrane or organelle of the first biological cell. In some instances, the second detectable marker is a fluorescent marker or an isotopic label, and in some instances, the second detectable marker labels a membrane or organelle or the second biological cell.

In some instances, the first biological cell and the second biological cell are from a subject. In some instances the first biological cell or the second biological cell comprise more than one detectable marker, and in some instances, the more than one detectable marker is a fluorescent marker or an isotopic label. In some instances, the more than one detectable marker labels a membrane or organelle of the first biological cell or the second biological cell.

In some instances, the genetic variation in the second genotype is engineered by a gene editing tool. In some instances, the gene editing tool is a transcription activator-like effector nuclease (TALEN), a zinc finger nuclease, or a CRISPR/Cas9. In some instances, the genetic variation is a genetic variation associated with a disease. In some instances, the genetic variation is a heterozygous genetic variation, and, in some instances, the genetic variation is a homozygous genetic variation.

In some instances, the plurality of vessels is at least 96 vessels, at least 384 vessels, at least 1,000 vessels, or at least 1,500 vessels. In some instances, a separate compound is provided in each vessel.

In some instances, the first biological cell is a mammalian cell and the second biological cell is a mammalian cell. In some instances, the first biological cell is a human cell and the second biological cell is a human cell. In some instances, the compound is a drug.

In some instances, the method further comprises determining the effect of the compound on the first biological cell and second biological cell. In some instances, the method further comprises determining the effect of the drug on the first biological cell and second biological cell.

Described herein are apparatuses for multiplexed screening, the apparatuses comprising: a microtiter plate; a first biological cell comprising a first detectable marker and a first genotype; a second biological cell comprising a second detectable marker and a second genotype, wherein the second genotype comprises a genetic variation relative to the first genotype; a compound; a first detection apparatus configured to detect the first detectable marker; and a second detection apparatus configured to detect the second detectable marker. In some instances, the apparatuses comprise a flow cytometer. In some instances, the first detection apparatus or the second apparatus comprises a mass spectrometer, an optical detector, or a microscope. In some instances, the first detection apparatus is the same as the second detection apparatus. In some instances, the microtiter plate comprises 384 wells.

In some instances, the first detectable marker is a fluorescent marker or an isotopic label, and in some instances, the first detectable marker labels a membrane or organelle of the first biological cell. In some instances, the second detectable marker is a fluorescent marker or an isotopic label, and in some instances the second detectable marker labels a membrane or organelle of the second biological cell.

In some instances, the first biological cell and the second biological cell are from a subject. In some instances, the first biological cell or the second biological cell comprises more than one detectable marker. In some instances, the more than one detectable marker is a fluorescent marker or an isotopic label. In some instances, the more than one detectable marker labels a membrane or organelle of the first biological cell or the second biological cell.

In some instances, the genetic variation in the second genotype is engineered by a gene editing tool. In some instances, the gene editing tool is a transcription activator-like effector nuclease (TALEN), a zinc finger nuclease, or CRISPR/Cas9. In some instances, the genetic variation is a genetic variation associated with a disease. In some instances, the genetic variation is a heterozygous genetic variation, and in some instances, the genetic variation is a homozygous genetic variation.

In some instances, the first biological cell is a mammalian cell and the second biological cell is a mammalian cell. In some instances, the first biological cell is a human cell and the second biological cell is a human cell. In some instances, the compound is a drug.

Described herein are kits comprising: a microtiter plate; a plasmid encoding a TALEN backbone; and instructions for performing the methods described herein. In some instances, the microtiter plate is pre-coated with a protein or a compound. In some instances, the kits further comprise an aliquot of a plurality of cells. In some instances, the kits further comprise an aliquot of an antibiotic. In some instances, the kits described herein further comprise a plasmid encoding a repeat variable diresidue. In some instances, the kits further comprise an aliquot of nucleotides.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a method of transcription activator-like effector nuclease (TALEN)-mediated cell labeling with a fluorescent marker. A safe harbor locus is identified (e.g., AAVS1), a donor DNA nucleic acid containing a fluorescent marker (with homology arms) is created, and TALENs specific to the safe harbor insertion site are provided, as in FIG. 1A. After insertion, the donor DNA with the fluorescent marker is integrated into the cell's DNA, and insertion is verified by PCR, as shown in FIG. 1B and FIG. 1C. FIG. 1D shows that successful integration of fluorescent marker is also verified by fluorescence microscopy.

FIG. 2 shows the fluorescence of three K562 clones after transcription activator-like effector nuclease (TALEN)-mediated cell labeling with a fluorescent marker. FIG. 2A shows the stable expression of red fluorescence protein (RFP) fluorescence by cells of clone A3 after targeted AAVS1 integration as shown by flow cytometry (top) and microscopy (bottom). FIG. 2B shows the stable expression of red fluorescence protein (RFP) fluorescence by cells of clone A8 after targeted AAVS1 integration as shown by flow cytometry (top) and microscopy (bottom). FIG. 2C shows the stable expression of red fluorescence protein (RFP) fluorescence by cells of clone B9 after non-targeted AAVS1 integration as shown by flow cytometry (top) and microscopy (bottom). FIG. 2D shows no fluorescence by K562 cells without AAVS1 integration by flow cytometry.

FIG. 3 shows variant frequency of R882H mutations introduced into K562 cells by TALENs. FIG. 3A shows Sanger sequencing of TALEN-edited K562 single clones in which the DNMT3A mutation was integrated into the cells (WT/WT), in which one copy of DNMT3A mutation (R882H/WT) was integrated into the cells, and in which two copies of DNMT3A mutation (R882H/R882H) was incorporated into the cells. FIG. 3B shows the variant frequency of TALEN-edited K562 cells, in which WT indicated the no integration of the DNMT3A mutation, NHEJ indicates integration of the DNMT3A mutation by non-homologous end joining, and HR indicates integration of the DNMT3A mutation by homologous recombination.

FIG. 4 shows a labeling strategy for distinguishing different populations of cells. FIG. 4A shows a membrane of a cell with a specific genotype labeled with mPLUM. FIG. 4B shows a nucleus of a cell with a different genotype than FIG. 4A labeled with mPLUM. FIG. 4C shows a membrane of a cell with a different genotype than FIG. 4A or FIG. 4B labeled with eGFP. FIG. 4D shows a nucleus of a cell with a different genotype than FIG. 4A, FIG. 4B, or FIG. 4C labeled with eGFP.

FIG. 5 shows how labeling the nucleus, the cell membrane, or the nucleus and the cell membrane with a combination of three different labels can lead to different unique labeling combinations for use in multiplexed screening.

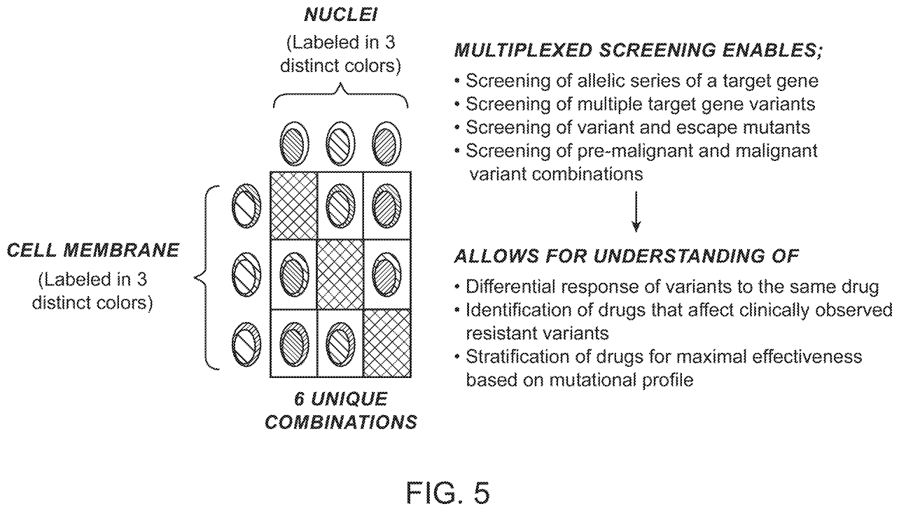

FIG. 6 shows a cytotoxicity assay in which cells are tested for viability in the presence of candidate compounds. Cells of different genotypes labeled with different detectable markers can be treated in co-culture with candidate compounds and used in a high-throughput (HT) imaging screen that assesses the number of viable cells after candidate compound treatment to evaluate the candidate compound's effect on viability of cells with different genotypes.

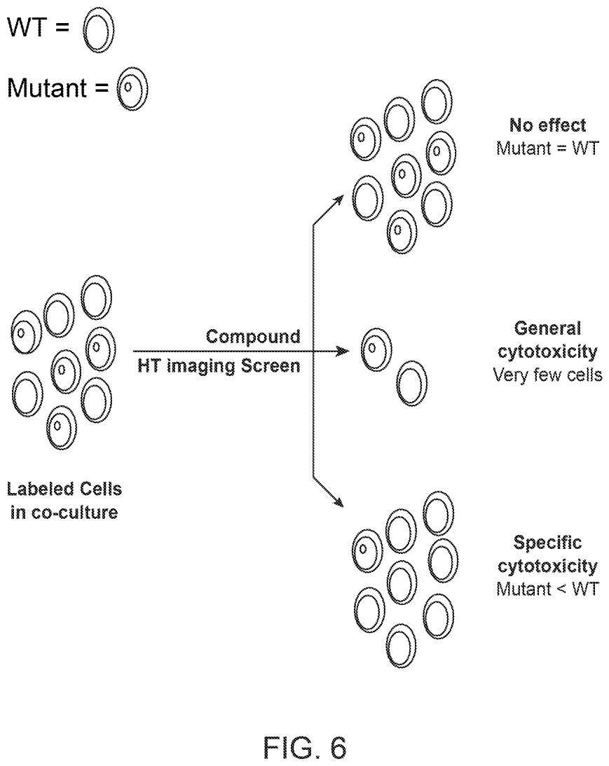

FIG. 7 shows flow cytometry data illustrating differences in surface marker expression by CD34-positive K562 cells with (R882H, light gray, rightmost panels) or without (WT, dark gray, rightmost panels) mutant copies of the DNMT3A gene. At Day 7, cell populations expressing CD41 and/or CD42 are approximately equal in number between wild-type (WT, leftmost panel) and mutant (DNMT3A R882H, center panel) K562 cells. At Day 14, a higher percentage of mutant cells are CD41-positive and CD42-negative compared to wild-type cell populations (illustrated in the overlay panels, found at the right of the figure). Since abnormal CD41 and CD42 expression kinetics are associated with the pathological differentiation of megakaryocytes and can be involved in the development of cancer, wild-type and/or mutant cell lines can be screened for abnormal maturation using detectable markers introduced by gene editing strategies.

FIG. 8 shows three different cell populations identified by organelle tags using the same fluorescent marker. FIG. 8A shows 786-O cells labeled by an organelle tracker dye that localizes to mitochondria. FIG. 8B shows a higher magnification of FIG. 8A, showing the specific pattern of dye localization to the mitochondria of a cell. FIG. 8C shows 786-O cells labeled by an organelle tracker dye that localizes to lysosomes. FIG. 8D shows a higher magnification of FIG. 8C, showing the specific pattern of dye localization to the lysosomes of a cell. FIG. 8E shows 786-O cells labeled by an organelle tracker dye that localizes to endoplasmic reticulum. FIG. 8F shows a higher magnification of FIG. 8C, showing the specific pattern of dye localization to the endoplasmic reticulum of a cell.

FIG. 9 shows different cell populations identified by organelle tags using the same fluorescent marker. FIG. 9A shows a mixed population of live 786-O cells that were separately labeled with specific organelle tracker dyes. FIG. 9B shows a higher magnification image of the mixed population of live 786-O cells with the same fluorescent marker from FIG. 9A, but in which the populations are distinguished by the localization of fluorescent marker to either the mitochondria, lysosomes, or the endoplasmic reticulum. Unlabeled cells were used as a negative control.

### DETAILED DESCRIPTION

The invention disclosed herein comprises methods and apparatuses that can be used to resolve a genetically heterogeneous population of cells in a single vessel. Techniques to improve resolution of co-cultured subpopulations described herein can include the genetic engineering of cell lines to include genetic variations, mutations, and/or alterations, which can include the expression of detectable labels, with which the different cellular subpopulations can be distinguished from one another. Experimental systems employing such methods and apparatuses can offer the advantages of minimizing vessel-to-vessel experimental variability and maximizing efficiency of experimental protocols and reagent usage.

As described herein, compound library screening can be conducted by multiplexed screening. The multiplexed screening can be a high-throughput multiplexed screening. The multiplexed screening can comprise using biological cells differentially labeled with detectable markers to distinguish their individual genotypes cultured in a vessel in the presence of a candidate compound, wherein the detectable markers can be assessed to identify how cells with different genotypes are affected by the candidate compound. For example, to determine if the candidate compound decreases the viability of a cell with a first genotype compared to a cell with a second genotype. The invention can be used in conjunction with a wide variety of detectable markers, such as fluorescent and isotopic labels, and those detectable markers can be used to specifically label a wide variety of membranes, organelles, and other structures related to the cells.

The methods described herein can include the use of a transcription activator-like effector nuclease (TALEN), zinc finger nuclease, or endonuclease system capable of recognizing a clustered regularly interspaced short palindromic repeat (CRISPR) to introduce a mutation, detectable marker, or both into the genotype of a cell. The detectable marker can be assessed using flow cytometry, optical microscopy, mass spectrometry, or a combination thereof. The presence, absence, distribution (e.g., pattern, localization, etc.), or intensity of a detectable marker can determine viability, proliferation, metabolic state, and/or differentiation of a cell. Cell types used in the invention can also include control cells (e.g., non-diseased or normal cells) to identify compounds that are less toxic or not toxic to non-diseased or normal cells, or are more efficacious in treating abnormal cell phenotypes relative to a cellular standard.

Therefore, a multiplexed screen as described herein can assess how the compound will affect both a diseased cell and a non-diseased or normal cell in a genetically heterogeneous population of cells due to these differential labeling and detection capabilities. The ability to differentiate a genetically heterogeneous population of cells in a single vessel can minimize variation from vessel to vessel, and provide for maximum similarity in conditions experienced by experimental and control cell groups. Furthermore, the amount of time and reagents required to carry out experiments can be minimized, as experimental and control cell groups are tested in a single vessel rather than two vessels. Distinguishing between cellular genotypes in a single imaging channel (via different localization patterns) frees up the other imaging channels to be used for discrimination of the measured biological phenotypes. Any disease or disorder that is associated with one or more genetic variations or mutations can be evaluated with the presently disclosed invention. A non-limiting list of diseases or disorders that are associated with one or more genetic variations or mutations can include any type of cancer, cardiovascular diseases or disorders, endocrine diseases or disorders, immune system diseases or disorders, hemic and lymphatic diseases or disorders, urogenital diseases or disorders, musculoskeletal diseases or disorders, nervous system diseases or disorders, metabolic diseases or disorders, otorhinolaryngologic diseases or disorders, respiratory tract diseases or disorders, skin and connective tissue diseases or disorders, neurodegenerative diseases or disorders, and stomatognathic diseases or disorders. Furthermore, a non-limiting list of diseases or disorders that are associated with one or more genetic mutations can include leukemia, bladder cancer, brain cancer, breast cancer, cervical cancer, colorectal cancer, esophageal cancer, liver cancer, lung cancer, skin cancer, ovarian cancer, pancreatic cancer, melanoma, lymphoma, prostate cancer, thyroid cancer, uterine cancer, bone cancer, throat cancer, congenital heart disease, multiple sclerosis, vasculitis, Alzheimer's disease, Parkinson's disease, dementia, muscular dystrophy, fibromyalgia, cystic fibrosis, and arthritis. A non-limiting list of diseases that are associated with genetic mutations that can be evaluated with the presently disclosed invention can also include acute myelogenous leukemia, chronic myelogenous leukemia, acute lymphocytic leukemia, chronic lymphocytic leukemia, light chain myeloma, non-secretory myeloma, multiple myeloma, Hodgkin lymphoma, and non-Hodgkin lymphoma.

Cells used in a Method Multiplexed Screening

A method of multiplexed screening can comprise screening a cell. The cell can be a biological cell and hereinafter is used interchangeably with the term “cell”. The cell can be an immortalized cell, such as a K562 leukemic cell. The cell can be a non-immortalized cell, such as a stem cell (e.g., an embryonic stem cell or an induced pluripotent stem cell (iPSC)). The cell can be derived from or differentiated from a stem cell. The cell can be a primary cell such as a human foreskin fibroblast. The cell can be derived from a subject. The cell can be a eukaryotic cell (e.g., animal, plant, algae, protozoa, or fungi). The cell can be a mammalian cell or non-mammalian cell (e.g., avian, reptilian, or insect). The cell can be from a non-human primate. The cell can be from a human. The cell can be a prokaryotic cell (e.g., bacteria). The cell can be a tumor cell, a cancer cell, or a cell from a specific tissue.

The cell can have a genotype that is not associated with a disease. The cell can have a genotype that is associated with a disease or biological trait (e.g., altered drug metabolism rate or susceptibility to drug toxicity). The cell can have a genotype that encodes a genetic variation associated with a disease or biological trait. The cell can have a genotype that encodes a genetic variation not associated with a disease or biological trait. The cell can have a genotype that encodes genetic variations associated with a disease or biological trait. The cell can have a genotype that encodes genetic variations not associated with a disease or biological trait. The cell can have a genotype that encodes a genetic variation associated with a disease and a genetic variation not associated with a disease or biological trait. A genetic variation associated with a disease can be any genetic variation that can lead to a physical manifestation or phenotype of the disease or biological trait. A genetic variation can be a mutation hereinafter is used interchangeably with the term “genetic variation”. The genetic variation can be a mutation in the nucleotide sequence of the genome of a first cell compared to the nucleotide sequence of the genome of a second cell. A genetic variation can be a single nucleotide variant or point mutation (e.g., substitution, insertion, or deletion) or a polynucleotide variant (e.g., substitutions, insertions, or deletions of at least two nucleotides). A genetic variation can be a silent, missense, nonsense, or frameshift mutation. A genetic variation can be a heterozygous mutation. A genetic variation can be a homozygous genetic variation. A genetic variation can be a hemizygous genetic variation. A genetic variation can be hypomorphic, hypermorphic, neomorphic, dominant-negative, haploinsufficient semi-dominant, gain-of-function, loss-of-function, or null.

The cell can be an unmodified cell. An unmodified cell can be a cell derived from a subject or from a cell line. An unmodified cell can be a cancer cell or diseased cell derived from a subject or from a cell line. An unmodified cell can be a non-diseased cell derived from a subject or from a cell line. An unmodified cell derived from a subject or from a cell line can comprise a genotype that is representative of a prevalent genotype of a specific population. An unmodified cell derived from a subject or from a cell line with a representative genotype can be referred to as a wild-type cell. An unmodified cell derived from a subject or from a cell line can contain an allele that is a representative allele of a specific population (e.g., a wild type allele). An unmodified cell derived from a subject or from a cell line with a representative allele can be referred to as a wild-type cell for the gene corresponding to that allele. Conversely, an unmodified cell derived from a subject or from a cell line can comprise a genotype that is not representative of a prevalent genotype of a specific population. An unmodified cell derived from a subject or from a cell line with an unrepresentative genotype can be referred to as a mutant cell or as a cell with a genetic variation. An unmodified cell derived from a subject or from a cell line can contain an allele that is not a representative allele of a specific population (e.g., a mutant allele). An unmodified cell derived from a subject or from a cell line with an unrepresentative allele can be referred to as a mutant cell for the gene corresponding to that allele or a cell with a genetic variation for the gene corresponding to that allele. In some cases, an unmodified cell derived from a subject can be expanded as a cell line and then can be used directly in the methods disclosed herein.

The cell can be a modified cell. A modified cell can be an unmodified cell that was mutated or genetically altered to comprise a genetic variation as compared to the unmodified cell. In some cases, an unmodified cell derived from a subject or cell line can be modified and expanded as a cell line, and then can be used directly in the methods disclosed herein. A cell can be modified once. A cell can be modified more than once. A cell can be modified multiple times simultaneously or in succession. For example, an established cell line, such as K562 (which has a previously existing mutation in exon 5), can be genetically edited to include a first detectable marker and then edited again to introduce a new mutation or genetic variation and a second detectable marker. The new mutation and second detectable marker can be expressed as a mutant protein fused to a second detectable marker or the mutation and the second detectable marker can be expressed individually. A cell can have 1, 2, 3, 4, 5, 6, 7, 8, 9, 10, or more modifications. The ability to alter a cell that has already been altered according to the methods as described herein can allow for increased customization of cellular reagents and an increased ability to distinguish between cells with different genotypes in the same culture.

An unmodified cell can be modified using gene editing strategies. Gene editing can allow customization of cell signaling, cell phenotype, and means of labeling, quantifying, and/or tracking cells. A cell can be modified by any of a number of strategies for gene editing. Gene editing can comprise introducing a mutation into the genome of a cell to modify its genotype. For example, a gene editing strategy can introduce a mutant or alternate nucleic acid sequences into a cell in a targeted manner to create a cell with a modified genotype. A cell can be modified by introducing a detectable marker using a gene editing strategy. A cell can comprise a mutation and a detectable marker. A gene editing strategy can comprise using a zinc-finger nuclease (ZFN), a transcription activator-like effector nuclease (TALEN), or a system involving an endonuclease targeted to clustered regularly interspaced short palindromic repeat (CRISPR). In some embodiments, homologous recombination can be used to introduce mutations into cells.

A ZFN can be an artificial restriction enzyme that can target a sequence of DNA through the ZFN's zinc finger DNA-binding domain and then can cause a double-strand DNA break via the ZFN's DNA-cleavage domain. By engineering a DNA-binding domain using a set of DNA-binding modules, selected from a library, that each correspond to a given three basepair sequence, ZFNs can be targeted to a specific region of DNA. The DNA-cleavage domain can consist of a type IIS restriction enzyme or the like (e.g., FokI), and is, in most embodiments, fused to the 5′ end of the DNA-binding domain via a linker sequence. The linker sequence can be between 5 and 7 basepairs in length. When used in pairs such that DNA-binding domains recognize sequences on opposite strands of a section of double-stranded DNA in such a way that the DNA-cleavage domains of the two ZFNs are aligned, a double-strand DNA break can be introduced in a targeted manner. In the presence of a section of repair template DNA containing a wild-type or mutated gene of interest, DNA repair mechanisms can either incorporate the template DNA at the location of the DNA break or can repair the DNA without incorporation.

A TALEN pair can be used to edit the genomes of cells used in the disclosed invention, wherein a TALEN can comprise a DNA cleavage domain and a transcription activator-like (TAL) effector DNA binding domain that can be customized to recognize specific nucleic acid sequences for the purpose of targeted genome editing. As in ZFN design, a TALEN design can involve a DNA-binding domain (known as a TALE) fused to a DNA-cleavage domain (Fold, for example) via a spacer, which, can be between 12 and 21 basepairs in length. A TALEN can be designed to recognize each strand of a double-stranded segment of DNA by engineering the TAL effector to include a sequence of repeat-variable diresidue subunits that can comprise approximately 28, 30, 32, 33, 34, 36, 38, 40 amino acid repeats capable of associating with specific DNA sequences, such that the DNA-cleavage domains of each TALEN align at the targeted DNA locus. A DNA template can be introduced before, immediately after or at the same time as the TALEN pair for incorporation at the site of the DNA break, which can be induced by the pair of TALENs' DNA-cleavage domains, by the cell's DNA repair mechanisms. Thus, TALEN-mediated gene editing can be used to introduce detectable markers into cells used in the disclosed methods and apparatus in a manner that allows for flexibility regarding DNA sequences that can be targeted for editing, further allowing for detectable markers to be specifically appended to endogenous nucleic acid sequences or placed under control of a similar promoter or enhancer as an endogenous gene of interest, and can further allow for faithful co-expression of the detectable marker by the same cell that is being studied. TALEN-edited sequences containing such expression of detectable markers under the control of a specific gene of interest's promoter or enhancer can also be accomplished using an artificial construct or non-integrating vector system, wherein detectable markers may not be permanently incorporated into the cell's genome.

Gene editing can also involve CRISPRs, in which one or more CRISPR-associated (Cas) endonucleases can be used to facilitate gene editing, including but not limited to Cas1, Cas2, Cas3, HD Cas3, Cas4, Cas5, Cas6, Cas7, Cas8, CARF, Csf1, Csn2, C2c1, C2c2, C2c3, Cpf1, RNaseIII, and DinG. In the case of CRISPR/Cas endonuclease gene editing, a guide RNA (gRNA) can be designed to associate directly with a DNA sequence of approximately 20 nucleotides in length, including sequences 15, 16, 17, 18, 19, 20, 21, 22, 23, 24 or 25 nucleotides in length, and to associate with a CRISPR-associated endonuclease, which can recognize a specific sequence of DNA that can be 3 nucleotides in length, known as a protospacer adjacent motif (PAM). Upon gRNA association with the target DNA sequence and Cas association with gRNA, the Cas endonuclease can create a single or double strand DNA break after recognizing the PAM sequence, thereby allowing for targeted genome editing. CRISPR/Cas systems can be used in pairs to remove a section of nucleotides from a given nucleic acid or they can be used to create targeted breaks in the DNA without the use of an additional CRISPR/Cas endonuclease pair to allow for insertion of custom nucleic acid sequences, such as nucleic acid sequences encoding a mutant (or wild type) gene variant or a detectable marker. Thus, the CRISPR/Cas system can be used to efficiently edit a cell's genomic material.

Targeted gene editing can serve as a means of introducing, deleting, or replacing a nucleic acid sequence. Using gene editing to introduce or to delete a nucleic acid sequence in a cell can alter which nucleic acids and proteins are produced in a cell. Introducing, deleting, or replacing a nucleic acid sequence through gene editing can also alter the quantity and/or function of nucleic acids and proteins produced in a cell, either directly (e.g., introduction of a mutation into a gene sequence) or indirectly (e.g., introduction or overexpression of a protein or nucleic acid such as a short hairpin ribonucleic acid (siRNA) or micro-ribonucleic acid (miRNA) that has the ability to bind, compete with, activate, or inactivate a molecule involved in a given signaling pathway or a molecule in a related signaling pathway). A genetic variation or mutation introduced into a cell can be a point mutation, insertion, deletion, or substitution, resulting in a silent, missense, nonsense, or frameshift mutation. Furthermore, the genetic variation or mutation can be a hypomorphic, hypermorphic, neomorphic, dominant-negative, haploinsufficient semi-dominant, gain-of-function, loss-of-function, or null mutation. Gene editing can also be used to induce the production of a new genetic product that would not normally be produced in a given cell (e.g., the production of a fluorescent protein from a nucleic acid sequence introduced into a cell or the production of a fusion RNA or protein that comprises a newly introduced nucleic acid or protein sequence appended onto a nucleic acid or protein sequence that is normally produced by the cell).

Introduction or replacement of a nucleic acid sequence in a cell through targeted gene editing (e.g., zinc-finger nuclease, transcription activator-like effector nuclease, homologous recombination, or clustered regularly interspaced short palindromic repeat-associated nuclease systems) can allow for the expression of custom nucleic acid sequences under endogenous circumstances. That is, nucleic acid sequences can be introduced or can replace a nucleic acid sequence in the genetic position that the endogenous nucleic acid sequence resides. This approach can ensure that genetic machinery (such as promoters, enhancers, silencers, repressors, and activators) that normally associate with the nucleic acid and/or its endogenous locus in the cell can access the introduced or replacement nucleic acid sequence. Introduction or replacement of a nucleic acid sequence into a specific genetic locus via gene editing can also maximize the probability that transcription and/or translation of an introduced or replacement nucleic acid can occur with similar kinetics and in a similar signaling sequence as nucleic acids in the same locus of an identical cell that has not been genetically edited or engineered. Thus, a nucleic acid sequence introduced by gene editing can be used to introduce custom a nucleic acid sequence into a cell such that the level of expression of that nucleic acid sequence is similar or identical to the level of expression of the nucleic acid sequence in the corresponding locus of an unaltered cell or in a corresponding unaltered locus in a similarly altered cell. Alternatively, a nucleic acid sequence introduced by gene editing can itself cause alterations in the level of expression of that nucleic acid sequence or other expressions levels of other nucleic acid sequences in the cell compared to levels of expression in an unaltered cell or in a similarly altered cell that contains the corresponding unaltered locus of that nucleic acid sequence.

Gene editing can also be used to stably introduce a nucleic acid sequence into a cell line. Stable introduction of a nucleic acid sequence into a cell line offers the advantage of creating an economically efficient, reproducible, and flexible platform with which to conduct the methods described herein. Variant cell lines can be created for different roles in the presently disclosed invention. For example, a reporter cell line can be created by introducing a nucleic acid sequence into a parent cell line and then individual sub-lines can be created via gene editing, each harboring different mutations, overexpressed sequences, and/or reporter genes. Additionally or alternatively, a modified cell can be a cell that is chemically mutated (e.g., through the pre-treatment with chemicals or compounds such as agonists, antagonists, altered oxygenation or pH, transfection reagents, permeabilizing agents, mitomycins, cytarabine, or enzymes), or physically mutated (e.g., through pre-treatment with increased or decreased temperature or with light or other forms of radiation such as visible or fluorescent light treatment, gamma irradiation, electrical field treatment, or magnetic field treatment). A genetic alteration to a cell can be spontaneous or induced (e.g., through gene editing or exposure to conditions that can alter genetic structure such as UV and ionizing radiation or crosslinking, dimerizing, or intercalating DNA reagents). Electroporation, lipofection, transfection, microinjection, viral transduction, and gene gun can be used to modify the genotype of a cell. Non-limiting examples of vector systems that can be used to introduce mutations into cells include viral vector, episomal vector, naked RNA (recombinant or natural), naked DNA (recombinant or natural), bacterial artificial chromosome (BAC), and RNA/DNA hybrid systems used separately or in combination. Vector systems can be used without additional reagents meant to aid in the incorporation and/or expression of desired mutations. A non-limiting list of reagents meant to aid in the incorporation and/or expression of desired mutations includes Lipofectamine, FuGENE, FuGENE HD, calcium phosphate, HeLaMONSTER, Xtreme Gene.

In some cases, these methods and/or vectors can be used to introduce material into a cell that does not alter the cell's genotype. Material introduced in this way can include RNA, DNA, RNA/DNA hybrids, proteins, or complexes of any of those molecules that have been assembled before introduction into the cell. In this way, introduction of these materials can be accomplished without directly affecting the cell's DNA. Introduction of material in this way can also be used as a means of transient intervention, since such materials can be degraded within the cell over time.

### Detectable Marker Labeling of Cells

A cell used in a multiplexed screen can be labeled by a detectable marker. A detectable marker can be a small molecule (e.g., a dye) or a macromolecule. A macromolecule can include polypeptides (e.g., proteins and/or protein fragments), nucleic acids, carbohydrates, lipids, macrocyles, polyphenols, and/or endogenous macromolecule complexes. The marker can be a distinguishable protein on the cell surface, in the cytoplasm, or localized to a specific cellular structure/organelle/biomolecule (e.g., the nuclear envelope, nucleoplasm, ribosomes, mitochondrial membranes, mitochondrial matrix, mitochondrial intermembrane space, actin, lamin, etc.). Alternatively, the detectable marker can be a secreted protein or a portion of a secreted protein. Cellular secretion (e.g., of hormones) can be studied with detectable markers by inhibiting secretion (e.g., with brefeldin A). Inhibition of cellular secretion (and subsequent evaluation of cellular secretion via detection of detectable markers) can be performed at a constant level for the duration of an experiment or it can be performed at one or more individual time points or over a time course. Such inhibition of cellular secretion can be administered to cells at a constant or variable dosage over a time course or at individual time points. Inhibition of secretion can allow for detection of the level of a protein labeled that would be normally be secreted by an individual cell.

A detectable marker can be an imaging agent. An imaging agent can include metals, radioisotopes, dyes, fluorophores, or any another suitable material that can be used in imaging. Detectable markers can be detected multiple times over the course of a procedure. Such serial measurements can be made to elucidate the differences in kinetics between cells of different genotypes with respect to signaling mechanisms, cellular morphology, changes in cellular phenotype or transcription, and viability.

A detectable marker can be an isotope or radioisotope. A molecule or precursor molecule used to label a cell can be labeled with a stable isotope or a radioisotope. Non-limiting examples of radioisotopes include alpha emitters, beta emitters, positron emitters, and gamma emitters. In some embodiments the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. Non-limiting examples of isotopes can be or other ²H, ¹³C, ¹⁵N, ¹⁸O, ³H, ¹⁴C, ³⁵S, ³²P, ³³P, ²¹⁵I, ¹³¹I, or other isotopes of elements that can be present in an organic system.

For isotopic labeling of a cell, an isotopically labeled precursor molecule can be incorporated into a cell by passage through a metabolic pathway in vivo in a living cell. Labeled precursor molecules can include, for example, H₂O, CO₂, NH₃, acetyl CoA (to form cholesterol, fatty acids); ribonucleic acids (to form RNA); deoxyribonucleic acid (to form RNA), deoxyribonucleic acid (to form DNA), glucose (to form glycogen), amino acids (to form peptides/proteins); phosphoenol-pyruvate (to form glucose/UDP-glucose); and glycine/succinate (to form porphyrin derivatives). The entire precursor molecule can be incorporated into one or more molecules in a metabolic pathway of a cell, or a portion of the precursor molecule can be incorporated into one or more molecules of interest within a cell. The isotope can be ³H, ¹⁴C, ³⁵S, ³²P, ³³P, ¹²⁵I, or ¹³¹I. For example, labeling with a protein precursor molecule can include introducing an amino acid, CO₂, NH₃, glucose, lactate, H₂O, acetate, and fatty acids incorporating an isotope into a cell. The precursor molecule can be one or more of ¹³C-lysine, ¹⁵N-histidine, ¹³C-serine, ¹³C-glycine, ²H-leucine, ¹⁵N-glycine, ¹³C-leucine, and any deuterated amino acid. An isotope labeled protein precursor can include, but is not limited to ²H-labeled amino acids, ¹³C labeled amino acids, ¹⁵N labeled amino acids, ¹⁸O labeled amino acids, ³³S or ³⁴S labeled amino acids, ³H₂O, ³H-labeled amino acids, and ¹⁴C labeled amino acids. A labeled amino acid can be administered, for example, undiluted or diluted with non-labeled amino acids. Organic metabolites, organic metabolite precursors, nucleic acids such as DNA or RNA, carbohydrates, lipids, or complex lipids can also be labeled with an isotope and introduced into a cell. The isotope can be ³H, ¹⁴C, ³⁵S, ³²P, ³³P, ¹²⁵I, or ¹³¹I.

A detectable marker can include a marker that can be detectable by a colorimetric method or a fluorescent method. For example, a colorimetric method can be an assay which utilizes reagents that undergo a measurable color change in the presence of an analyte (e.g., an enzyme, an antibody, a compound, a hormone). Exemplary colorimetric methods can include enzyme-mediated detection method such as tyramide signal amplification (TSA) which utilizes horseradish peroxidase (HRP) to generate a signal when digested by tyramide substrate and 3,3′,5,5′-Tetramethylbenzidine (TMB) which generates a blue color upon oxidation to 3,3′5,5′-tetramethylbenzidine diamine in the presence of a peroxidase enzyme such as HRP. A detectable marker described herein can include a marker that can be detectable by a colorimetric method.

A detectable marker can also include a marker that can be detectable by a fluorescent method. The detectable marker can be a marker expressed by a modified or unmodified cell to express the detectable marker. The modified or unmodified cell can express this detectable marker on the cell's surface, which can then be detected by a fluorescent marker. The modified or unmodified cell can express this detectable marker within the cell, which can then be detected by a fluorescent marker. The detectable marker can be a fluorescent marker. A fluorescent marker can be a small molecule (e.g., a dye) or a fluorescently labeled macromolecule. A fluorescently labeled macromolecule can include a fluorescently labeled polypeptide (e.g., a labeled protein and/or a protein fragment), a fluorescently labeled nucleic acid molecule, a fluorescently labeled carbohydrate, a fluorescently labeled lipid, a fluorescently labeled macrocyle, a fluorescently labeled polyphenol, and/or a fluorescently labeled endogenous macromolecule complex (e.g., a primary antibody-secondary antibody complex).

A fluorescent small molecule can comprise rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, thiorhodamine, cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, proflavin, acridine orange, acridine yellow, arylmethine derivatives, auramine, crystal violet, malachite green, tetrapyrrole derivatives, porphin, phtalocyanine, bilirubin 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), 5(6)-FAM, 5-FAM, Fluorescein dT, 5-TAMRA-cadavarine, 2-aminoacridone, HEX, JOE (NHS Ester), MAX, TET, ROX, TAMRA, TARMA™ (NHS Ester), TEX 615, ATTO™ 488, ATTO™ 532, ATTO™ 550, ATTO™ 565, ATTO™ Rho101, ATTO™ 590, ATTO™ 633, ATTO™ 647N, TYE™ 563, TYE™ 665, or TYE™ 705.

A fluorescent marker can comprise Cy3, Cy5, Cy5.5, Cy7, Q570, Alexa488, Alexa555, Alexa594, Alexa647, Alexa680, Alexa 750, Alexa 790, Atto488, Atto532, Atto647N, TexasRed, CF610, Propidium iodide, Q670, IRDye700, IRDye800, Indocyanine green, Pacific Blue dye, Pacific Green dye, Pacific Orange dye, green fluorescent protein (GFP), enhanced green fluorescent protein (eGFP), fluorescein isothiocyanate (FITC), Clover, yellow fluorescent protein (YFP), blue fluorescent protein (BFP), cyan fluorescent protein (CFP), red fluorescent protein (RFP)), Discosoma sp. red fluorescent protein (dsRed), m isoform proteins and any derivative thereof (such as, for example, mCherry, mPlum, mStrawberry, mKate2, mEmerald, and mNeonGreen), or Hoeschst stains and any derivative thereof.

A label can be applied to a cell in a number of ways, including antibody-mediated labeling, direct conjugation, genetic encoding (as a separate or fusion protein), and incorporation via culture additives (e.g., isotopic labeling through culture additives). In some embodiments involving expression of labels from one or more nucleic acid sequences, labels can either take the form of a fusion protein, in which the label is physically connected to one or more proteins translated from RNA or to one or more proteins transcribed from DNA or cDNA and then translated from mRNA, or they can take the form of a separate protein, which is produced in the cell from RNA, DNA, or cDNA either in conjunction with another gene or gene segment (for example, separated by a 2A skip sequence) or on its own and is not physically connected to another protein immediately after it is created. However, overexpression of fluorescent protein tags can be hampered by artefactual results and transient and variable protein expression. Therefore, integration of the fluorescent protein gene cassette into a defined genomic locus that is known to sustain physiological levels of expression (e.g., a “safe harbor”) can produce cells that exhibit consistent, reproducible, and sustained levels of fluorescent protein expression within a narrow range. This can be accomplished with highly efficient cleavage at an endogenous safe harbor locus, such as AAVS1, and integration (and subsequent expression) of a fluorescent protein gene cassette, such as mCherry and eGFP.

In some embodiments, the labeled protein can be incorporated into the structure of an organelle or other cellular structure or molecular complex, thus specifically labeling that organelle, structure, or complex. An organelle can be labeled by an organelle tracker dye that localizes to an organelle, such as localizing to mitochondria, lysosomes, or endoplasmic reticulum. Different organelles can be labeled with the same detectable marker. Different organelles can be labeled with different detectable markers. In other embodiments, the labeled protein is not permanently incorporated into any organelle, structure, or complex but can be associated with or otherwise temporarily incorporated into one or more organelle, structure, or complex for the purpose of qualitative or quantitative analysis involving those organelles, structures, or complexes. In other embodiments, free labels produced during the same transcription or translation event as the protein of interest (or under the control of a similar promoter) can be evaluated quantitatively or qualitatively to assess the presence or extent of pathway activity.

A detectable marker to be used to label a cell can be introduced to the cell exogenously. Exogenous introduction of a detectable marker can involve, for example, using an antibody to label a cell's surface, using a dye to label a cellular compartment or structure (e.g., a lipophilic dye such as DiI, DiO, DiD, DiA, or DiR to label a cell membrane or a live/dead dye such as calcein AM and/or ethidium homodimer-1, which may involve host cell enzymes or substrates in the process of generating or suppressing a detectable marker signal). Detectable markers can also be introduced exogenously by “feeding” the detectable marker or tracker to the cell. For example, a detectable marker may introduced to the cell through phagocytosis, pinocytosis (e.g., diffusion or convection of small detectable markers through cell pores or cell channels, such as aquaporins), or receptor-mediated endocytosis. In some embodiments, the detectable marker may comprise the means for inducing receptor-mediated endocytosis.

A first cell can be labeled with a detectable marker to distinguish it from a second cell. In some embodiments, the first cell comprising a first genotype can be labeled with a first detectable marker and a second cell comprising a second genotype can be labeled with a second detectable marker. The first genotype of the first cell can comprise a genetic variation or mutation as compared to the second genotype of the second cell. The first detectable marker and the second detectable marker can be the same detectable marker, but the first detectable marker can be differentially located in a cell compared to the second detectable marker. The first detectable marker and the second detectable marker can be different detectable markers. Detectable markers can be used to uniquely label each genetically unique cell population cultured in a single culture vessel, allowing for co-culture of at least 2, at least 3, at least 4, at least 5, at least 6, at least 7, at least 8, at least 9, at least 10, at least 15 cell lines or cell types in a single culture vessel. Moreover, detectable markers can be used to label individual proteins, aggregates, structures, membranes, or organelles associated with the cell, allowing for further classification, quantification, characterization, and tracking of individual populations and subpopulations of co-cultured, as each unique combination of detectable markers can allow distinguishing populations and/or subpopulations from other populations and subpopulations, even in a high-throughput or multiplexed mode of experimentation. The use of several types of labels, either within the same class of label (e.g., multiple fluorescent labels with different fluorophores) or between more than one class of label (e.g., the use of a fluorescent label in conjunction with an isotopic label) can also be used.

In some embodiments, the cell can be engineered (e.g., with TALEN-mediated genome editing) to express an “anchor” molecule (such as a protein) with which an exogenously applied detectable marker may associate. For example, a cell or cell line used in experimentation can be engineered to express a molecule not normally expressed by that cell or cell lines such that the molecule is expressed on the cell's surface. The molecule incorporated into the experimental cell system can be selected for its ability to specifically interact with a detectable marker or molecule associated with a detectable marker such that only cells engineered in this way are labeled with the detectable marker. Thus, this strategy can be used in the methods and systems described herein to label or stain individual cells or cell lines with a specific detectable marker.

### Multiplexed Screening

A method of multiplexed screening can comprise providing a plurality of vessels, wherein each vessel can contain cells with detectable markers cultured with a compound, and then the detectable markers are detected in each vessel. In some embodiments, the method of multiplexed screening comprises providing a plurality of vessels, wherein each vessel comprises a first biological cell comprising a first detectable marker and a first genotype; and a second biological cell comprising a second detectable marker and a second genotype, wherein the second genotype comprises a genetic variation or mutation relative to the first genotype; contacting the first biological cell and the second biological cell with a compound in each vessel; and detecting the first detectable marker and the second detectable marker after the contacting in each vessel. In some embodiments the compound is a drug. In further embodiments, the effect of the drug on the first cell is compared to the effect on the second cell can be determined. This effect can be comparing phenotype, functionality, and viability of the first cell compared to the second cell. The method can be performed by an apparatus comprising a microtiter plate; biological cells comprising detectable markers; a compound; and a detection apparatus configured to detect detectable markers. In some embodiments, the method can be performed by an apparatus comprising a microtiter plate; a first biological cell comprising a first detectable marker and a first genotype; a second biological cell comprising a second detectable marker and a second genotype, wherein the second genotype comprises a mutation relative to the first genotype; a compound; a first detection apparatus configured to detect the first detectable marker; and a second detection apparatus configured to detect the second detectable marker.

### Vessels

A vessel can be a well in a microtiter plate. A plurality of vessels can be a plurality of wells in a microtiter plate. A microtiter plate can be a flat plate comprising multiple wells or vessels that can be used as to culture cells. A microtiter plate can contain 6, 24, 96, 384, or 1536 wells arranged in a rectangular matrix. Each well or vessel of a microplate can hold a certain volume of liquid. This volume of liquid can be between tens of nanoliters of liquid to several milliliters of liquid. The bottom of the well or vessel can be flat or round. The shape of the well or vessel can be circular or square. The surface of the well or vessel can be modified using an oxygen plasma discharge to make the surface more hydrophilic for tissue culturing. The more hydrophilic surface can be an easier surface for adherent cells to grow on. Other coatings (e.g., such as poly-L-lysine, collagen, laminin, etc.) can be utilized to render cells that are usually grown in suspension to become more adherent. Such adherent culture of normally suspension-cultured cells can be useful for convenience of imaging. A microtiter plate can be made of polystyrene, polypropylene, cyclo-olefin, or polycarbonate. A microtiter plate can be designed for handling by a robot. A robot can aspirate or dispense liquid samples from or to plates, transport the microtiter plate between instruments, incubate the microtiter plate, or be involved in detecting specific biological, chemical, or physical characteristics of a cell population in the wells or vessels.

### Cells in Each Vessel

A vessel can contain a cell labeled with a detectable marker. A vessel can contain a biological cell labeled with a detectable marker. A vessel can contain a plurality of cells. A vessel can contain a plurality of biological cells. The plurality of cells can be cells with different genotypes. The vessel can comprise at least two cells, wherein a first cell has a first genotype and a second cell has a second genotype. The second genotype can be a modified genotype of the first genotype. A cell can be modified by any of the strategies previously described, including strategies for gene editing. Gene editing can comprise introducing a mutation into the genome of a cell to modify its genotype. For example, a gene editing strategy can introduce a mutant or alternate nucleic acid sequences into a cell in a targeted manner to create a cell with a modified genotype. A gene editing strategy can comprise using a zinc-finger nuclease (ZFN), a transcription activator-like effector nuclease (TALEN), or a system involving an endonuclease targeted to clustered regularly interspaced short palindromic repeat (CRISPR). A cell can be modified by introducing a detectable marker using a gene editing strategy. A cell can comprise a mutation and a detectable marker. In some embodiments, homologous recombination can be used to introduce mutations into cells. The modification of the second genotype can be a heterozygous mutation in a gene as compared to the first genotype. The modification of the second genotype can be a homozygous mutation in a gene as compared to the first genotype. The modification of the second genotype can be any of the modifications as previously described. The modification of the second genotype can be a mutation in multiple genes as compared to the first genotype. The modification of the second genotype can be 1, 2, 3, 4, 5, 6, 7, 8, 9, 10, or more mutations compared to the first genotype.

Additionally, the first cell can be labeled with a first detectable marker, and the second cell can be labeled with a second detectable marker. A detectable marker can be any of the detectable markers previously described, such as an imaging agent. An imaging agent can be a metal, radioisotope, dye, fluorophore, isotope, or any another suitable material that can be used in imaging. A detectable marker can be an isotope or radioisotope. A first cell can be labeled with one or more detectable markers. A second cell can be labeled with one or more detectable markers. The one or more detectable markers of the first cell can be different than the one or more detectable markers in a second cell. A label can be applied to a cell in a number of ways, including antibody-mediated labeling, direct conjugation, genetic encoding (as a separate or fusion protein), and incorporation via culture additives (e.g., isotopic labeling through culture additives). Additionally, the labeled protein can be incorporated into the structure of an organelle or other cellular structure or molecular complex, thus specifically labeling that organelle, structure, or complex. For example, an organelle tracking dye can be used to label an organelle. The same color organelle tracking dye can be used to label different organelles. In other embodiments, the labeled protein is not permanently incorporated into any organelle, structure, or complex but can be associated with or otherwise temporarily incorporated into one or more organelle, structure, or complex for the purpose of qualitative or quantitative analysis involving those organelles, structures, or complexes. In other embodiments, free labels (e.g., detectable markers or labels not associated with another protein or structure but present in the cytoplasm or nucleoplasm) produced during the same transcription or translation event as the protein of interest (or under the control of a similar promoter) can be evaluated quantitatively or qualitatively to assess the presence or extent of pathway activity. Brefeldin A, or any similar reagent that prevents cellular secretion, can be added to a vessel with the first cell and the second cell to prevent secretion of free labels, and therefore allow for the detection of the free labels.

A vessel can contain a plurality of cells that are a genetically heterogeneous population of cells. As used herein, genetic heterogeneity can refer to genomic heterogeneity (e.g., cells from different subjects, or cells harboring different mutations), epigenetic heterogeneity (e.g., cells that express different genes, different levels of genes, or have different epigenetic modifications), and/or phenotypic heterogeneity (e.g., cells from different tissues, different tumors, or different subjects). Each genotype in a genetically heterogeneous population of cells can be a modified genotype of the other cell genotypes. The modification can be a specific mutation in a gene. The gene can be a gene associated with a disease. A genotype can have 1, 2, 3, 4, 5, 6, 7, 8, 9, 10, or more mutations compared to the other genotypes. The mutation can be heterozygous or homozygous. The mutations can be introduced into cells using gene editing strategies previously described. The genetically heterogeneous population can be a mixed population of at least 2, 3, 4, 5, 6, 7, 8, 9, 10, 15, 20, 25, 30, 35, 40, 45, 60, 55, 60, 65, 70, 75, 80, 85, 90, 95, 100, or more cell genotypes. The cells can be the same cell type or different cell types. The cells can have the same or different endogenous genetic backgrounds. The cells can be from one subject or from multiple subjects.

Furthermore, a vessel can contain a plurality of cells that are a genetically heterogeneous population of cells, wherein each cell of the same genotype is labeled with the same detectable marker, and each genotype can be identified by the type or presence/absence of detectable marker, the pattern of the detectable marker signal, or localization of the detectable marker with respect to cellular structures or other detectable markers. In some embodiments, each cell of the same genotype can be labeled with the same combination of detectable markers, and therefore each genotype in the genetically heterogeneous population can be identified by a different combination of detectable markers. A detectable marker can be any detectable marker as previously described herein, such as an imaging agent. Some non-limiting examples of an imaging agent can include a metal, radioisotope, dye, fluorophore, isotope, or any another suitable material that can be used in imaging. A detectable marker can be an isotope such as ³H, ¹⁴C, ³⁵S, ³²P, ³³P, ¹²⁵I, or ¹³¹I. For example, in a genetically heterogeneous population, a first cell can be labeled with an isotope and a second cell can be labeled fluorophore.

In some embodiments, different cellular groups (e.g., cell lines of different genotypes) can be labeled with the same detectable marker (e.g., the same fluorophore, such as eGFP) directed toward or fused to the same molecule (e.g., a mitochondrial molecule), and the different cellular groups can be distinguished based on the reproducibly detectable marker signal intensity, signal pattern, or signal localization. For example, a mutant cell may exhibit punctate staining of a given detectable marker-labeled molecule while a wild type cell may exhibit diffuse staining of the same detectable marker-labeled molecule. In this way, a wild type cell line labeled with a detectable marker directed toward, for example, a mitochondrial protein can be distinguished from a mutant cell line labeled with the same detectable marker directed toward the same mitochondrial protein wherein the shape and/or size of the mitochondria is visibly different in the mutant cell than the shape and/or size (e.g., morphology) of the mitochondria of the wild type cell line. Another non-limiting example includes the use of detectable markers directed to the same protein, which is localized differently within the cell in a wild type cell line as compared to a mutant cell line (e.g., a molecule that fails to translocate to the nucleus if the nuclear localization signal for that molecule has been mutated). In some aspects, the methods and systems described herein can involve the use of detectable markers that can be detected in the same detection channel (e.g., the same spectrum detection range of a fluorescent microscope detector, which can be defined by the user prior to, during, or after image acquisition) and directed toward molecules normally located in separate compartments of the cell (e.g., a detectable marker fusion protein localized exclusively to the cell's surface and a detectable marker fusion protein localized exclusively to the nucleus or antibodies that recognize molecules that are similarly distinguishable). In this way, it is possible to make more detection channels available for the application of additional detectable markers. That is, by using one detectable marker channel to identify both wild type and mutant cells (e.g., through pattern of labeling, intensity of labeling, or localization within the cell), the additional detectable marker channels that might have been required to uniquely label each different cell group can be used to interrogate other aspects of the cell with additional detectable markers, allowing more parameters to be evaluated in each experiment.

In some embodiments, the discrimination of cell lines (based on, for example, type, location, intensity, or presence or modulation over time of detectable marker(s)) wherein the cells are labeled with the same detectable marker directed to the same molecule of interest can be performed manually by the user, and any additional detectable markers or aspects of the data can be assigned to the appropriate cell group, cell line, genotype, treatment condition, or phenotype. In some embodiments, computer executable software can be provided in which images of the cells obtained during experimentation can be analyzed using a computer program, and additional parameters quantified from the remaining detectable markers present (or a subset thereof) can be assigned to the appropriate cell line, cell group, genotype, treatment condition, or phenotype by the program.

Quantification or qualitative analysis of images and/or detectable marker signal(s) can be used for molecular profiling or cell tracking. Molecular profiling can involve labeling a sample with a set of target-specific imaging probes or detectable markers such that a cell is identified by a particular detectable marker or a particular set of two or more detectable markers. A molecular description of various cell types or cell states (e.g., phenotypic states, such as metabolic states, mitotic/meiotic states, activation states, etc.) can be used to define or identify cells or phenotypic states in experimental samples. Alternatively or simultaneously, cells can be quantitatively or qualitatively tracked between measurements or time points by analyzing the set of detectable markers present in a sample and/or associated with a cell, either in homogeneous or heterogeneous experimental samples, which can involve quantitative or qualitative analysis of each cell independent of other cells or specifically in relation to other cells in the same sample or in other samples, which may or may not involve different experimental conditions. These analyses can be performed manually by the experimenter or automatically by a computer program designed for such analytical functions.

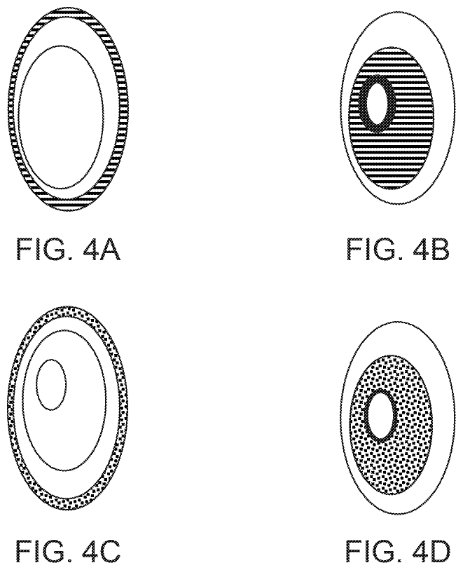

Additionally, the labels can be applied to or expressed by a cell in a number of ways, including antibody-mediated labeling, direct conjugation, genetic encoding (as a separate or fusion protein), and incorporation via culture additives (e.g., isotopic labeling through culture additives), or a labeled protein can be incorporated into the structure of an organelle or other cellular structure or molecular complex, thus specifically labeling that organelle, structure, or complex(e.g., lysosomes, mitochondria, individual portions of the golgi apparatus, nuclear membrane, chromatin, vacuole, autophagosome, centrosome, cytoskeleton or the endoplasmic reticulum). The first detectable marker and the second detectable marker can be different detectable markers. For example, the nucleus of a first cell with a first genotype can be labeled with a first detectable marker and the plasma membrane of a second cell with a second genotype can labeled with a second detectable marker. Furthermore, a combination of detectable markers can be used to identify multiple different genotypes in a genetically heterogeneous population of cells. For example, a genetically heterogeneous population with six different genotypes can be labeled with three distinct fluorophores by labeling the different organelles such as the nucleus and the plasma membrane with different combinations of the fluorophores. FIG. 4 illustrates how this can result in 6 unique label combinations for identifying each genotype.

Alternatively, the first detectable marker and the second detectable marker can be the same detectable marker, in which the two genotypes of the labeled cells can be differentiated by the cellular localization of the detectable marker or organelle morphology labeled with the detectable marker. For example, a detectable marker can be the same fluorescent marker, which can be used to label a first organelle of a cell with a first genotype and can be used to label a second organelle of a cell with a second genotype. As another example, heterogeneous mixture of multiple cell genotypes can be labeled by the same detectable marker, in which the detectable maker localizes to a different organelle for each genotype. Some non-limiting examples of different organelles that can be labeled with a detectable marker that localizes to that organelle are lysosomes, mitochondria, individual portions of the golgi apparatus, nuclear membrane, chromatin, vacuole, autophagosome, centrosome, cytoskeleton, or the endoplasmic reticulum. The subsequent pattern of detectable marker organelle localization can be used to distinguish between cells of different genotypes. The ability to differentiate between different cell genotypes based on the organelle localization pattern of the same detectable marker can allow for the use of additional detectable markers for determining biological readouts of each cell genotype. For example, fluorescently labeled Lamin A/C antibodies or CellLight fluorescent nucleus probes can be used for assaying nuclear integrity, CellMask plasma membrane stain can be used for assaying cell plasma membrane, or RedoxSensor Red CC-1 can be used for assaying the oxidative state of the cell cytoplasm in addition to the detectable marker used for differentiating cell genotypes.

The cells can be co-cultured together in the same vessel. The cells can proliferate at different rates, interact with one another directly or indirectly, or tolerate alterations to culture conditions (e.g., culture medium and additives, substrates, temperature, oxygenation, etc.) to a differing degree, with respect to phenotype, functionality, and viability. These changes can be measured against a control cell type. A control cell type can include a cell related to experimental cell types by a similar genetic background (e.g., a genetic background that can be identical to experimental cell types or that can be derived from experimental cell types, as in cells from the same donor or cell line in which one or more mutations have been introduced) or can be independent of experimental cell types with respect to genetic background.

In some embodiments, there can be multiple vessels composed of the same genetically heterogeneous population of cells. In other embodiments, there can be multiple vessels composed of different genetically heterogeneous populations of cells. The multiple vessels can be in a microtiter plate. The multiple vessels can be in multiple microtiter plates. Use of multiple microtiter plates can allow for a large or numerous experimental group and/or multiplexing of experimentation.

### Multiplexed Screening of a Compound

A compound can be added to a vessel comprising a first biological cell comprising a first detectable marker and a first genotype; and a second biological cell comprising a second detectable marker and a second genotype, wherein the second genotype comprises a genetic variation relative to the first genotype. The compound can contact the first biological cell in the vessel. The compound can contact second biological cell in the vessel.

A compound can be a drug. A compound can be a small molecule. The compound can be a novel small molecule. The compound can be a previously known small molecule. A compound can be a peptide or peptidomimetic molecule. A compound can be nucleic acid. A compound can be DNA or RNA. The compound can be from a chemical library or compound library, e.g., a natural product or member of a combinatorial chemistry library. A given library can comprise a set of structurally related or unrelated compounds. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and can include those such as the “split and pool” or “parallel” synthesis techniques, solid-phase and solution-phase techniques, and encoding techniques (see, for example, Czarnik, Curr. Opin. Chem. Bio. 1:60-6 (1997)). The compound can be a compound from a commercially available small molecule library.

Multiple compound libraries can be used in the screening of compounds. For example, an initial library of compounds (e.g., at least 10,000 compounds, at least 15,000 compounds, at least 20,000 compounds, or at least 25,000 compounds), representing the diversity of chemical compounds in a given chemical space or subspace (which can include chemical compounds of, for example, similar chemical structure or physiochemical characteristics), can be used as an initial screen for structural or functional (e.g., phenotypic) effect(s) of the compounds on a cell or cell line. The cells can also be used to screen one or more additional compound libraries of a smaller (e.g., no more than 10,000 compounds, no more than 5,000 compounds, no more than 2,500 compounds, no more than 1,000 compounds, or no more than 500 compounds) or larger size (e.g., at least 50,000 compounds, at least 100,000 compounds, at least 200,000 compounds, at least 250,000 compounds, at least 300,000 compounds, at least 500,000 compounds, at least 750,000 compounds, at least 1 million compounds, at least 1.5 million compounds, at least 2 million compounds, or at least 3 million compounds) representing a more diverse chemical space, a more focused chemical space (e.g., more selective in terms of number or chemical subspace diversity), or a chemical space of similar breadth or scope. Subsequent compound library screens may be performed sequentially to assay compounds in more diverse or more focused chemical spaces and subspaces or to assay compounds in a chemical space of similar scope. The selection of compound libraries, both in terms of size and chemical space diversity or focus, can be based upon data from previous screens or known characteristics of individual compounds and/or compound classes in a given space. Compound libraries containing 2-3 million compounds (e.g., “full deck” libraries) can be used as a means of agnostic (e.g., unbiased) screening of compounds.

Additionally, more than one compound can be added to a vessel. A combination of 1, 2, 3, 4, 5, 6, or more different compounds can be added to a vessel.

A different compound or combination of compounds can be added to each vessel of a plurality of vessels comprising the same genetically heterogeneous population of cells in each vessel. Each different compound or combination of compounds can be from a chemical or compound library. In some embodiments, the plurality of vessels can be in a multi-titer plate. The multi-titer plate can comprise 6, 24, 96, 384, or 1536 wells or vessels.

### Detecting Cells in a Vessel

A cell in a vessel can be detected by a detectable marker. A molecule or structure associated with a cell can also be detected by a detectable marker. The detectable marker can identify the genotype of the cell. The cell can be detected after contacting a compound. A plurality of cells can be detected by the cells' detectable markers. The detectable markers can identify the genotype of each cell. The detectable marker can be detected by any detection apparatus capable of detecting the detectable marker. For example, a detectable marker can be detected by an optical detector. In some embodiments, a fluorescent detectable marker can be detected by a flow cytometer. In other embodiments, a fluorescent detectable marker can be detected by a microscope. Alternatively, an isotope detectable marker can be detected by a mass spectrometer, mass spectrometer microscope, or mass cytometer (e.g., with isotopically pure rare earth elements). Additionally, a combination of detection apparatuses can be used to detect multiple detectable markers in a cell or a plurality of cells. In a multiplexed assay, two or more cells with detectable markers and different genotypes can be present in a given vessel and distinguished from one another through various means of detection including, but not limited to, stimulation and optical detection of fluorescent markers, mass spectrometric detection of isotopic markers, optical detection of physical characteristics of the cells, or any combination thereof. Detection of cells harboring fluorescent markers can comprise stimulating cells in microtiter plate vessels with light of a wavelength capable of exciting the fluorophore such that it emits photons within that fluorophore's theoretical emission spectrum and recording those emitted photons using an optical detector. Detection of cells harboring detectable markers while in culture can occur at any time point and can occur at one or multiple time points during experimentation (e.g., when observing changes in cells or their behavior over a time course).

By culturing different populations of cells in the same vessel, subtle variations in metrics between the different cell populations can be better detected and assessed due to the preclusion of experimental error that occurs when detecting cells from different vessels. For example, experimental error can be introduced when setting thresholds of detection (e.g., when defining detector gain for measurement of fluorescent detectable markers), which are then used for detection of cells in different vessels. This can lead to differences in background signal between different vessels, therefore obscuring the detection of subtle variations in the effect of the compound on each cell genotype, such as the expression level (e.g., of protein or RNA), cell viability, phenotype, or functionality between different populations of cells.

The invention can also comprise various experimental interventions during and following culture of cellular components. These interventions can take place prior to or during collection of data. Cells can be altered or otherwise stimulated with chemical or physical stimuli. Cells can be treated or pre-treated with heat or cold shock, hypoxia, hyperoxia, increased or decreased pH, physical stimuli (e.g., light or other forms of radiation such as visible or fluorescent light treatment, gamma irradiation, electrical field treatment, magnetic field treatment, as well as tensile, compressive, or shearing forces administered in cyclical or acute patterns), differentiation factors, or compounds such as agonists, antagonists, transfection reagents, permeabilizing agents, mitomycins, cytarabine, or enzymes. Cells can be chemically fixed or cryogenically treated prior to detection of detectable markers. In some embodiments, cells can be fixed and enzymatically or mechanically removed from culture (or removed from culture and then fixed) prior to be subjected to one or more methods of detection (e.g., microscopy, flow cytometry, mass spectrometry, etc.). Cells can be permeabilized prior to detection using a permeabilization agent (e.g., detergents such as Triton X-100, Tween 20, saponin, organic solvents such as methanol and acetone, etc.). If additional detectable agents are to be used to label cells (e.g., affinity tags such as antibodies conjugated, fused, or otherwise associated with a detectable marker), the additional detectable agents may be added to the cells before or after permeabilization. If a given detectable marker or detectable agent is on the cell's surface or if the detectable agent is produced inside of the cell, permeabilization is not required.

### Microscopy

A detectable marker can be detected by optical microscopy using a microscope. The microscopy can be fluorescence microscopy. Fluorescence microscopy can be two-photon or multi-photon imaging.

The presence or absence of a detectable marker can be detected by microscopy. Furthermore, microscopy can be used to detect the localization of a detectable marker in an organelle or plasma membrane of a cell. For example, microscopy can be used to detect a fluorophore localized in the nucleus of a cell.

In some instances, one or more far-field fluorescence techniques can be utilized for the detection or localization of detectable makers. In some instances, a microscopy method can be a high magnification oil immersion microscopy method. In such method, the wide-field and confocal fluorescent microscopes can achieve sub-cellular resolution. In some instances, a microscopy method can utilize a super-resolution microscopy, which allows images to be taken with a higher resolution than the diffraction limit. A super-resolution microscopy method can include deterministic super-resolution, which utilizes a fluorophore's nonlinear response to excitation to enhance resolution. Exemplary deterministic super-resolution can include stimulated emission depletion (STED), ground state depletion (GSD), reversible saturable optical linear fluorescence transitions (RESOLFT), and saturated structured illumination microscopy (SSIM). A super-resolution microscopy method can also include stochastic super-resolution, which utilizes a complex temporal behavior of a fluorophore, to enhance resolution. Exemplary stochastic super-resolution method can include Super-resolution optical fluctuation imaging (SOFI), all single-molecular localization method (SMLM) such as spectral precision determination microscopy (SPDM), SPDMphymod, photo-activated localization microscopy (PALM), FPALM, stochastic optical reconstruction microscopy (STORM), and dSTORM.

In some cases, a microscopy method can be a single-molecular localization method (SMLM). In some instances, a microscopy method can be a spectral precision determination microscopy (SPDM) method. A SPDM method can rely on stochastic burst or blinking of fluorophores and subsequent temporal integration of signals to achieve lateral resolution at, for example, between about 10 nm to about 100 nm.

In some cases, a microscopy method can be a spatially modulated illumination (SMI) method. A SMI method can utilize phased lasers and interference patterns to illuminate specimens and increase resolution by measuring the signal in fringes of the resulting Moire patterns.

In additional cases, a microscopy method can be a synthetic aperture optics (SAO) method. A SAO method can utilize a low magnification, low numerical aperture (NA) lens to achieve large field of view and depth of field, without sacrificing spatial resolution. For example, an SAO method can comprise illuminating the detection agent-labeled target (e.g., a regulatory element) with a predetermined number (N) of selective excitation patterns, where the number (N) of selective excitation patterns is determined based upon the detection agent's physical characteristics corresponding to spatial frequency content (e.g., the size, shape, and/or spacing of the detection agents on the imaging target) from the illuminated target, optically imaging the illuminated target at a resolution insufficient to resolve the objects on the target, and processing optical images of the illuminated target using information on the selective excitation patterns to obtain a final image of the illuminated target at a resolution sufficient to resolve the objects on the target. In some instances, the number (N) of selective excitation patterns corresponds to the number of k-space sampling points in a k-space sampling space in a frequency domain, with the extent of the k-space sampling space being substantially proportional to an inverse of a minimum distance (4×) between the objects that is to be resolved by SAO, and with the inverse of the k-space sampling interval between the k-space sampling points being less than a width (w) of a detected area captured by a pixel of a system for said optical imaging. In additional cases, the number (N) can include a function of various parameters of the imaging system (e.g., magnification (Mag) of the objective lens, numerical aperture (NA) of the objective lens, wavelength λ_{E} of the light emitted from the imaging target, and/or effective pixel size p of the pixel sensitive area of the CCD, etc.).

In some cases, a SAO method can analyze a set of detection agent profiles from at least 100, 200, at least 230, at least 250, at least 500, at least 1000, or more cells imaged simultaneously within one field of view utilizing an imaging instrument. The one field of view can be a single wide field of view allowing image capture of greater than 100, greater than 200, greater than 230, greater than 500, greater than 1000, or more cells. The single wide field of view can be about 0.70 mm by about 0.70 mm field of view. The SAO imaging instrument can enable a resolution of about 0.25 μm with a 20×/0.45NA lens. The SAO imaging instrument can enable a depth of field of about 2.72 μm with a 20×/0.45NA lens. The imaging instrument can enable a working distance of about 7 mm with a 20×/0.45NA lens. The imaging instrument can enable a z-stack of 1 with a 20×/0.45NA lens. The SAO method can further integrate and interpolate 3-dimensional images from 2-dimensional images.

In some instances, a SAO imaging instrument is an SAO instrument as described in U.S. Publication No. 2011/0228073 (Lightspeed Genomics, Inc).

A cell can be chemically fixed or cryogenically treated prior to detection of detectable markers by microscopy. In some embodiments, cells can be fixed and enzymatically or mechanically removed from culture (or removed from culture and then fixed) prior to detection.

Different cell populations can be differentiated by labeling each cell population with a tracking dye specific for different organelles. The dye can label different organelles using the same color. After detection by microscopy, image processing and algorithm analysis using a computer can be used to differentiate the patterns of localization associated with the localization of a dye to a certain organelle. For example, parameters for fluorescence can be defined for each organelle, thus allowing a computer to differentiate the fluorescent pattern in a cell that indicates dye localization to that organelle. This can be used to differentiate different cell populations labeled by different organelles tagged with the same fluorescent dye.

### Flow Cytometry

Flow cytometry can be used to screen cells that are not adherent (e.g., cells normally cultured in suspension) or cannot be made adherent (e.g., through the use of vessel coatings) for a detectable marker. Flow cytometry can be used to screen cells that can be detached from adherent culture for a detectable marker. Cells can be sorted using a flow cytometer as part of analyses such as sequencing or quantitative polymerase chain reaction (qPCR). A detectable marker can be detected by flow cytometry using a flow cytometer. A detectable marker detected by flow cytometry can be a fluorescent marker. For example, a fluorescent marker can comprise rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, thiorhodamine, cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, proflavin, acridine orange, acridine yellow, arylmethine derivatives, auramine, crystal violet, malachite green, tetrapyrrole derivatives, porphin, phtalocyanine, bilirubin 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), 5(6)-FAM, 5-FAM, Fluorescein dT, 5-TAMRA-cadavarine, 2-aminoacridone, HEX, JOE (NHS Ester), MAX, TET, ROX, TAMRA, TARMA™ (NHS Ester), TEX 615, ATTO™ 488, ATTO™ 532, ATTO™ 550, ATTO™ 565, ATTO™ Rho 101, ATTO™ 590, ATTO™ 633, ATTO™ 647N, TYE™ 563, TYE™ 665, or TYE™ 705. Additionally, a fluorescent marker can comprise Cy3, Cy5, Cy5.5, Cy7, Q570, Alexa488, Alexa555, Alexa594, Alexa647, Alexa680, Alexa 750, Alexa 790, Atto488, Atto532, Atto647N, TexasRed, CF610, Propidium iodide, Q670, IRDye700, IRDye800, Indocyanine green, Pacific Blue dye, Pacific Green dye, Pacific Orange dye, green fluorescent protein (GFP), enhanced green fluorescent protein (eGFP), fluorescein isothiocyanate (FITC), Clover, yellow fluorescent protein (YFP), blue fluorescent protein (BFP), cyan fluorescent protein (CFP), red fluorescent protein (RFP)), Discosoma sp. red fluorescent protein (dsRed), m isoform proteins and any derivative thereof (such as, for example, mCherry, mPlum, mCerulean, mStrawberry, mKate2, mEmerald, and mNeonGreen), Hoeschst stains, or any derivative thereof.

Flow cytometry can be a laser-based, biophysical technique that can be used for cell counting, cell sorting, and biomarker detection. A flow cytometer is a detection apparatus that can perform a simultaneous multiparametric analysis of the physical and chemical characteristics of thousands of particles per second in a stream of fluid comprising cells in a suspension as it passes by an electronic detector.

A flow cytometer can comprise a flow cell, a measuring system, a detector and Analogue-to-Digital Conversion (ADC) system, an amplification system, and a computer. A flow cell can carry and align cells in a liquid (i.e., in sheath fluid) so that the cells can pass single file through the light beam of the flow cytometer for sensing. A measuring system can be used to measure the impedance (or conductivity) and optics in the system, and can comprise lamps such as mercury and xenon, high-power water cooled lasers such as argon and krypton, low-powered air-cooled lasers such as argon (488 nm), red-HeNe (633 nm), green-HeNe, HeCd (UV), or diode lasers such as blue, green, red, or violet. A detector and ADC system can be used to convert the analogue measurements of forward-scattered light, side-scattered light, and dye-specific fluorescence signals into measurements that can be processed by a binary computer. An amplification system can be linear or logarithmic. A computer can be used for analysis of the signals and data collected.

Flow cytometers can have multiple lasers and fluorescence detectors. For example, a flow cytometer can have 1, 2, 3, 4, 5, 6, 7, 8, 9, or 10 lasers, and 1, 2, 3, 4, 5, 6, 7, 8, 9, 10, 11, 12, 13, 14, 15, 16, 17, or 18 fluorescence detectors. This can allow for the detection of multiple fluorescent markers in the same sample.

A specialized type of flow cytometry, referred to as Fluorescence-Activated Cell Sorting (FACS), can be used to separate and isolate cells having specific characteristics. FACS can allow for the sorting of a heterogeneous mixture of cells into two or more containers. This can occur one cell at a time. A cell can be sorted upon its specific light scattering and fluorescent characteristics. For example, a cell is sorted from a cell suspension by flowing the cells suspension in the center of a narrow, rapidly flowing stream of sheath fluid. The flow can be arranged so that there is a large separation between cells relative to cell diameter. A vibrating mechanism can cause the stream of cells to break into individual droplets comprising one cell per droplet. Before the stream breaks into droplets, the flow passes through a fluorescence measuring station where the fluorescence of each cell of interest is measured. An electrical charging ring can be placed just at the point where the stream breaks into droplets. A charge can placed on the ring based immediately prior to fluorescence intensity being measured, and then the opposite charge can be trapped on the droplet as it breaks from the stream. The charged droplet can then fall through an electrostatic deflection system that diverts the droplet into a container based upon its charge. In some systems, the charge can be applied directly to the stream, and the droplet breaking off can retain charge of the same sign as the stream. The stream can then be returned to neutral after the droplet breaks off.

Therefore, a first cell with a first detectable marker can be sorted from genetically heterogeneous population of cells comprising other detectable markers. Other detectable markers in the cell can then be detected using other detection techniques as described herein. For example, the sorted cell can be analyzed by mass spectrometry for an isotope detectable marker.

A cell can be chemically fixed or cryogenically treated prior to detection of detectable markers by flow cytometry. In some embodiments, cells can be fixed and enzymatically or mechanically removed from culture (or removed from culture and then fixed) prior to detection. The detectable marker can be on the cell surface (i.e., the membrane does not need to be permeabilized) or an intracellular marker (i.e., the membrane needs to be permeabilized).

### Mass Spectrometry

A detectable marker can be detected by various methods of mass spectrometry, including but not limited to gas chromatography-mass spectrometry (GC-MS), isotope-ratio mass spectrometry, GC-isotope ratio-combustion-MS, GC-isotope ratio-pyrrolysis-MS, liquid chromatography-MS, electrospray ionization-MS, matrix assisted laser desorption-time of flight-MS, Fourier-transform-ion-cyclotron-resonance-MS, and cycloidal-MS. A detectable marker detected by mass spectrometry can be an isotope. An isotope used as a detectable marker can be ²H, ¹³C, ¹⁵N, ¹⁸O, ³H, ¹⁴C, ³⁵S, ³²P, ³³P, ¹²⁵I, ¹³¹I, or other isotopes of elements that can be present in an organic system.

A mass spectrometer can convert molecules into rapidly moving gaseous ions and separate them on the basis of their mass-to-charge ratios. The distributions of isotopes or iso-topologues of ions, or ion fragments, can thus be used to measure the isotopic enrichment of a cell.

Generally, a mass spectrometer can include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization. Techniques for ionization of large, non-volatile macromolecules including proteins and polynucleotides can include electrospray ionization (ESI) and matrix assisted laser desorption (MALDI). These can be applied in combination with sample separation introduction techniques, such as liquid chromatography and capillary zone electrophoresis.

In addition, two or more mass analyzers can be coupled (MS/MS) first to separate precursor ions, then to separate and measure gas phase fragment ions. These instruments can generate an initial series of ionic fragments of a protein, and then can generate secondary fragments of the initial ions. The resulting overlapping sequences can allow complete sequencing of the protein, by piecing together overlaying “pieces of the puzzle”, based on a single mass spectrometric analysis within a few minutes (plus computer analysis time).

In addition, mass spectrometers can be coupled to separation means such as gas chromatography (GC) and high performance liquid chromatography (HPLC). In gas-chromatography mass-spectrometry (GC/MS), capillary columns from a gas chromatograph can be coupled directly to the mass spectrometer, optionally using a jet separator. In such an application, the gas chromatography (GC) column can separate sample components from the sample gas mixture and the separated components can be ionized and chemically analyzed in the mass spectrometer.

### Additional Methods of Quantification

Analysis of experimental reagents (e.g., cells, supernatant, purified molecules and molecular complexes) can also include techniques such as quantitative polymerase chain reaction (e.g., for measurement of gene expression levels), affinity purification, affinity detection (e.g., Western blotting, enzyme-linked immunosorbent assays, fluorescence in situ hybridization, and immunoprecipitation), Southern blotting (e.g., for monitoring induction of mutations using radiation therapy), sequencing (e.g., Sanger sequencing and next gen sequencing), and analysis of post-translational modifications using mass spectrometry, Western blotting, or ELISAs. Use of one of these assays alone or in combination with other assays or techniques described herein can further elucidate information about cells of interest, such as the mechanism of action of a given candidate compound, rate of cellular metabolism, or the mechanism of progression of an abnormal condition (e.g., disease state) in a cell of a specific genotype or phenotype.

### Timing of Detection

The detection of detectable markers can be made pre-addition of a compound, at addition of a compound, post-addition of a compound, or any combination thereof. The detection can be made at time intervals pre-addition of a compound, post-addition of a compound, or both. The intervals can be regular time intervals or irregular time intervals.

Detectable markers can serve as a coding system for distinguishing between cell genotypes or phenotypes in a genetically heterogeneous population of cells in a vessel. As a non-limiting example, wild type cells labeled with one isotopic marker and mutant cells labeled with a second isotopic marker can be used to determine overall cytotoxic effects of applied candidate compounds using mass spectrometric detection after a specified incubation time, while fluorescent detectable markers expressed by wild type and mutant cells, or by cells with or without a genetic variation relative to each other, such that cells of each genotype express a different detectable marker, can be detected by optical imaging at one or more time points during culture to monitor cell number and proliferation rate in culture. In some embodiments, selection and design of such a panel of detectable markers comprises customizing gene editing strategies (e.g., TALEN-, zinc-finger nuclease-, or CRISPR/Cas-mediated gene editing) to place expression of the fluorescent detectable markers under the control of a promoter or enhancer of a gene associated with proliferation (e.g., cyclin-dependent kinases).

As such, the selection of detectable markers and the procurement of cells labeled with those detectable markers, which can include the introduction of a detectable marker into cells so that it is specifically associated with a given protein or structure in the cell through, for example, TALEN-mediated genetic engineering, can be made with the intention of providing a set of detectable markers that can distinguish cells of different genotype or phenotype during execution of the methods described herein. For example, over the course of multiplexed differentiation assays, cells can pass through a transient phenotypic state in which a certain protein labeled with a detectable marker is temporarily expressed, indicating a subpopulation of, e.g., normally differentiating wild type cells that may not be present in mutant cells, and this detectable marker can be used to distinguish wild type cells that are in the process of normal differentiation from wild type cells that are not, for whatever reason, in that stage of differentiation and from mutant cells that may not be capable of entering that stage of differentiation.

Data from Multiplexed Screening

Multiple metrics of a cell in a genetically heterogeneous population of cells at a single time point or over a range of time points and for culture or co-culture of cells prior to, between, or after data collection time points can be assessed. In some embodiments, this includes the detection of one or more cellular labels, one or more morphological metrics, one or more functional metrics, or any combination thereof while cells are cultured or co-cultured in microtiter plate vessels. Metrics measured, recorded, and/or analyzed in this invention can be directed toward assessing the effects of compounds on cells with different genotypes, the compound's effects on cellular metabolism, the cellular metabolism of a compound, exposure to stimuli (e.g., chemical or physical stimuli), and time-dependent variables (e.g., the retention of cellular markers related to immature phenotypes over time in culture). Examples of metrics that can be measured, recorded, and/or analyzed in this invention (i.e., the effects of the compound or other conditions as described herein) can include cell viability, proliferation rate, metabolic rate and function, organelle integrity and organization, cellular maturation or differentiation, and cellular function (e.g., protein production, enzymatic activity, migration, exertion of mechanical force, and phagocytic function).

The method of detection of data can be a high-throughput screen, which can screen through hundreds, thousands, tens of thousands, hundreds of thousands, or millions of vessels or wells on microtiter plates. This can comprise using robotics, data processing and control software, liquid handling devices, and sensitive detectors to take, process, and analyze measurements taken from each well of a microtiter plate. The apparatus used for the high-throughput screen can output the resulting measurements as a grid of numeric values, with each number mapping to the value obtained from a single well. A high-capacity analysis machine can measure dozens of plates in the space of a few minutes to quickly generate thousands of experimental data points.

Over the course of experimentation employing the methods and apparatus disclosed herein, data collection can be made for all vessels or a subset of vessels used in the invention and the cells contained therein at regular or irregular intervals, including before, during, at the conclusion, and after the conclusion of experimentation. Data can be collected from individual wells one or more times during the use of the invention, and the method of data collection can, in some embodiments, differ between data collection time points for each vessel or between vessels at each time point. Many of the cellular labels compatible with this invention can be detected through conventional means, but data collection can also entail assessment of cell characteristics that are not related to cellular labels. For example, a non-limiting list of means of data collection can include fluorescence microscopy to detect the presence of a fluorescent marker for a specific cellular marker or indicating cellular viability, cell tracking to assess cell migration and proliferation (e.g., optical image capture of cell boundaries and positions), optical imaging to assess cellular morphology, pH metering using colorimetric indicators, sequencing of cellular or cell-free experimental reagents using high-throughput sequencing technology (e.g., Sanger sequencing and next gen sequencing), or methods of spectrometry, such as mass spectrometry and nuclear magnetic resonance (NMR) spectrometry to detect isotopic labeling or molecular characteristics. All of this data can be matched with the detectable marker(s) of the cell that indicate the genotype of the cell, which can be referred to as the detectable marker code.

Data collection can involve assessing cell viability. Assessments of cell viability can involve detection of detectable markers or evaluation of cellular characteristics, such as cell and organelle morphology (as determined by means which include light microscopy, fluorescence microscopy, structured light microscopy, or electron microscopy), population doubling time, migration, and adherence in culture. Viability assessment using one or more detectable marker(s) can involve collecting data pertaining to the detectable markers' intensities or spatial positions (relative to other structures or detectable markers in the same cell or relative to other cells or detectable markers in the same vessel), at a single time point or over several measurements. Events that can be measured and collected as data for analysis of cellular viability include cellular spilling associated with necrosis, vacuolization, and blebbing. Detectable markers can be indicative of a normal cellular function (a detectable marker under the control of a promoter associated with protein secretion in a mature cell type) or an abnormal cellular function (e.g., a detectable marker under the control of a promoter associated with apoptosis). The absence of detectable markers can also be collected as data, if such an absence can be attributed to a particular cellular state (e.g., the destruction or inhibition of a fusion mRNA encoding a detectable marker through the function of a micro RNA or siRNA or the quenching of a detectable marker by a chemical intervention or through exposure to a type of radiation). For example, a fluorescent detectable marker under the control of a promoter known to function only in viable cells can be quenched by exposure to laser light, and the rate at which fluorescent signal returns can be related to cell viability. Therefore, the effects of a treatment with a compound or other contextual event (cumulative or acute) on cell viability can be determined.

Toxicity of a compound, stimulus (e.g., exogenous stimulus, chemical stimulus, physical stimulus, or any combination thereof), or culture reagent can be assessed by examining data collected for the purpose of assessing cell viability. Toxicity of a compound, stimulus, or culture reagent can be determined by cell metrics before, after, or during exposure to a compound, stimulus, or culture reagent; for example, by recording changes in the number of adherent cells in culture or the presence of viability or apoptotic markers over time in the presence or absence of a compound. Toxicity can also be assessed by considering other metrics independently or in conjunction with one another, such as changes in or absolute values of proliferation rate, metabolic rate and function, organelle integrity and organization, cellular morphology, maturation or differentiation, and cellular function (e.g., protein production, enzymatic activity, migration, exertion of mechanical force, and phagocytic function).

Cell proliferation can similarly be assessed during data collection. Data regarding cell proliferation and cell proliferation rates, like cell viability, can be collected by measuring detectable markers (with respect to intensity, spatial position, or temporal co-presentation with another measurable aspect of the assay) or by analyzing aspects of cellular morphology or functionality. In some embodiments, cellular proliferation (as with other metrics, like cell viability, differentiation, and metabolic rate) can be assessed by different means at different time points (e.g., proliferation may be determined by comparing cell number determined at one time point using light microscopy and an edge-finding computer algorithm and compared to a cell number determined at a later time point using counts of a given detectable marker). Cellular proliferation can be assessed by measuring cell number or number of cell divisions over time (which can be accomplished, for example, by using manual or automated means to quantify cellular structures or detectable markers such as gene edited detectable markers, intercalating detectable markers like Hoescht dyes, or cell stains like bromodeoxyuridine or 4′,6-diamidino-2-phenylindole) or by comparing these measurements with other cells or another cell population. Determinations of cell proliferation can be made in light of other experimental conditions, such as the addition of differentiation factors to the culture or the addition of an experimental compound to the culture. In this way, the effect of contextual factors, like co-culture with other cells, changes in culture pH or temperature, or the addition of a compound to the cell culture, on cell proliferation can be determined from data collected during experimentation.

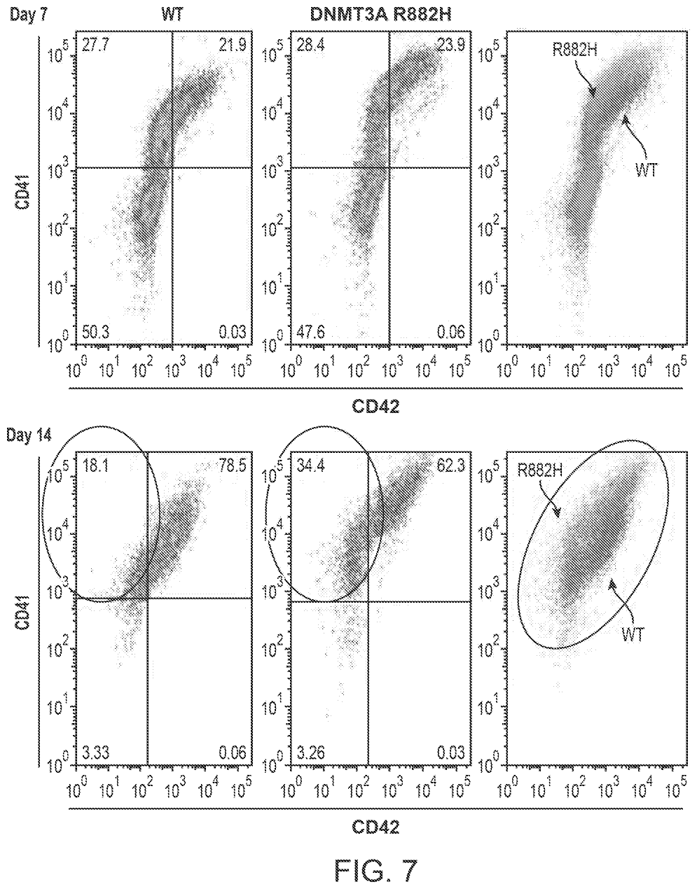

Maturation or differentiation of cells in culture can be assessed by data collected during experiments. Maturation and differentiation can be processes involving changes to cell function, morphology, and/or signaling kinetics that can help to distinguish the phases of progression as a progenitor cell (which can also be known as stem cells or immature cells) becomes a specialized cell type. Improper progression through the phases of maturation (e.g., establishing a more robust phenotypic profile for a given specialized cell type) and/or differentiation (e.g., the process of defining a cellular lineage or progressing through the cell type intermediates of that cell lineage) can lead to dysfunction at the tissue, organ, or organism level; therefore, collection of data regarding these processes can inform conclusions made in screening assays. Furthermore, detectable markers can be made to signify aspects of cellular processes related to cellular maturation or differentiation (e.g., the presence, production, or phosphorylation of a molecule of interest), for example, by being co-expressed in the cell or by being expressed as part of a fusion protein with the molecule of interest. Data collection can include evaluation of detectable markers, secreted substances, and morphological characteristics of cells in culture, as expression of certain nucleic acids or proteins and changes in cellular function can be indicative of transitions between progenitor phases or cellular specialization. One example of a detectable marker being used to identify a process of maturation in a cell can be an eGFP-CD42 fusion protein during megakaryocyte maturation (as in FIG. 7), as R882H DNMT3A mutant K562 leukemic cells may not be expected to transition to a CD42-positive state at the same rate as wild-type K562 leukemic cells.

A cell's metabolic profile can influence both normal cellular function and cellular dysfunction. In some situations, a cell's metabolic profile can be altered by conditions present in a given experiment, such as the addition of a drug compound. If a drug causes cellular metabolism to decrease, it can, in certain circumstances, be an indication that the drug is toxic to the cell at the dosage in question. In other situations, a lack of change in the metabolic profile of a cell in response to the addition or increased dosage of a compound can indicate drug tolerance or ineffectiveness. In some cases, a drug can affect a cell's metabolism while its enantiomer does not affect the cell's metabolic profile. Measurements of a cell's metabolic profile can include quantification of molecule production, molecule degradation, or production of metabolites. Measurements of metabolic profile can also include evaluation of glycolysis, oxidative respiration, spatial localization of proteins relative to organelles and structures such as the mitochondria (e.g., using detectable markers and/or specific measurement systems like an Agilent Seahorse Extracellular Flux (XF) Analyzer). Since metabolic profile metrics may reach the same levels between groups by the end of experimentation, it may be necessary to compare groups using measurements recorded at intermediate time points and to compare groups as a whole, across an entire time course (e.g., using a two-way analysis of variance or two-way ANOVA). Thus, data regarding the metabolic profile of a cell can be collected at various time points and/or for various concentrations of a given treatment compound or compounds as a means of determining drug efficacy and any selective drug metabolism. For example, a compound can be used to treat a co-culture of mutant and wild-type cells in a multiplexed drug screening assay, and data can be collected regarding whether the compound had a deleterious effect on one cell type, the other cell type, or both cell types by monitoring each cell's metabolic profile over the course of the experiment. Compounds found to specifically affect the viability of mutant cells (e.g., cancer cells harboring a p.R882H mutation in the DNMT3A gene) while leaving wild type cells unaffected may be considered more promising candidate drugs than those negatively affecting both mutant and wild-type cells.

Collecting data over multiple regularly spaced or irregularly spaced time points can provide additional insight into the effects of a given candidate compound or into the response of a given cell. In some instances, different cell populations can be expected to reach the same endpoint with respect to a given metric by a certain point in the experimental protocol. In such situations, informative results can be present prior to that point in the experimental protocol and can comprise the rate at which cells of each group reach the common endpoint for that metric. Thus, it can be informative to collect data at intermediate time points over the course of the experiment. In certain cases, the kinetics of a given cell reaching such a common endpoint for a given metric can be linear, and, in some cases, it can be nonlinear. Therefore, it can be useful to concentrate measurements for the metric in question near the point in experimentation when nonlinear kinetics are expected to occur (e.g., an exponential growth phase or the “toe” region of a nonlinear curve) so that the details of the nonlinear region are captured and unnecessary measurements can be avoided. Rate of onset (or loss) of phenotype can be an experimental metric that can be considered when determining sampling rates for experimental measurements in a given application of the invention.

In some cases, the metrics captured and/or analyzed using the methods described herein can be used to make inferences and conclusions regarding a specific patient or a population of patients. The use of cells derived from a particular patient or a representative portion of a patient population can be used to assess the response of those cells to experimental interventions, such as treatment with a candidate compound. Accordingly, data collected during experimentation regarding cellular response (e.g., cell viability, proliferation, metabolic rate, specific toxicity, etc.) to experimental conditions can be used to determine not only prioritization of candidate compounds in a clinical setting but to determine aspects of treatment regimens such as drug dosage amounts and durations as well. Furthermore, depending on the source of the cells used and the genetic modifications made, these results can be used to inform treatment regimens for individual patients and populations of patients.

Detection of quantifiable metrics (which includes the detection of detectable markers as well as measurements made regarding cellular structures and using visible light microscopy) can be numerical counting or quantification of a signal's intensity. That is, measurements can be made such that the presence, absence, or relative number of a detectable marker, cellular structure, or other quantifiable aspect of an experiment can be recorded. Detection of metrics and markers can also be made with respect to the degree to level of detection (e.g., the intensity of a signal), even when they are being quantified in terms of presence or absence. Thus, intense signals can be discriminated from weak signals of the same marker in a different cell, even when the number of signals are the same and are located in a similar position, spatially, with respect to the cell.

### Detectable Marker Code

Detectable markers can be suitable for use as an encoding platform for distinguishing cells with different genotypes in multiplexed screening. In some aspects, the present disclosure can provide combinations of detectable markers capable of encoding and/or biomolecular encoding. In some aspects, a specific detectable marker or combination of detectable markers can be a detectable code, also referred to herein as a “code” or “encoding.” In other aspects, a specific detectable marker or combination of detectable markers can be an optically detectable code, also referred to herein as an “optical code” or “optical encoding.” The “code” or “optical code” can enable a cell with that detectable marker or combination of detectable markers to be distinguished from cells having a different code, and thus a different genotype. Various combinations of encoding schemes can suitable for use with the encoded cells described herein. In some aspects, the detectable code can include measuring qualitative or quantitative amounts of an isotope, such as ²H, ¹³C, ¹⁵N, ¹⁸O, ³H, ¹⁴C, ³⁵S, ³²P, ³³P, ¹²⁵I, ¹³¹I, or other isotopes of elements that can be present in an organic system. In certain aspects, the optically detectable code can include one or more optical properties of fluorescent detectable markers, such as a predetermined emission spectrum of a fluorescent detectable marker (e.g., emission wavelength, emission intensity), a predetermined emission lifetime of the fluorescent detectable marker, a predetermined emission rate, a predetermined absorption wavelength, or a combination thereof. Furthermore, the optically detectable code can include localization of the fluorescent detectable markers, which can be based on the morphology of the localization, such as localization to the plasma membrane, organelles such as the nucleus, or a combination thereof. Accordingly, an encoded cell can be uniquely identified by measuring its isotopic properties, optical properties, or a combination thereof, in order to determine the corresponding code.

In other aspects, the encoded cell can include one or more isotopic detectable markers that used to define the code. In some aspects, an encoded cell can include one or more distinct fluorescent detectable markers that are used to define the optically detectable code. In still other aspects, an encoded cell can include one or more distinct fluorescent detectable markers localized to specific organelles that are used to define the optically detectable code. The encoded cell can include any suitable number and combinations of distinct detectable markers, such as only a distinct detectable marker, two or more distinct detectable markers, three or more or more distinct detectable markers, four or more distinct detectable markers, five or more distinct detectable markers, six or more distinct detectable markers, seven or more distinct detectable markers, eight or more distinct detectable markers, nine or more distinct detectable markers, ten r more distinct detectable markers, twenty or more distinct detectable markers, fifty or more distinct detectable markers, or one hundred or more distinct detectable markers.

In some aspects, the localization of a detectable marker to a morphologically distinct cellular compartment or organelle can be used to distinguish different encoded cells. In some aspects, an encoded morphological localization can include two or more, three or more, four or more, five or more, six or more, seven or more, eight or more, nine or more, or ten or more distinct morphological localizations that are distinguishable from each other.

In certain aspects, distinct detectable markers can have one or more optical properties (e.g., emission spectra, emission intensities, emission wavelengths, emission lifetimes, emission rates, absorbance wavelengths, etc.) that are distinguishable from one another. In some aspects, an encoded detectable marker can include two or more, three or more, four or more, five or more, six or more, seven or more, eight or more, nine or more, or ten or more distinct detectable markers having emission spectra that are distinguishable from each other. In some aspects, the encoded detectable markers can include two or more, three or more, four or more, five or more, six or more, seven or more, eight or more, nine or more, or ten or more distinct detectable markers having emission intensities that are distinguishable from each other. In some aspects, the encoded detectable markers can include two or more, three or more, four or more, five or more, six or more, seven or more, eight or more, nine or more, or ten or more distinct detectable markers having emission wavelengths that are distinguishable from each other. In some aspects, the encoded detectable markers can include two or more, three or more, four or more, five or more, six or more, seven or more, eight or more, nine or more, or ten or more distinct detectable makers having emission lifetimes that are distinguishable from each other. In some still other aspects, the encoded detectable markers can include two or more, three or more, four or more, five or more, six or more, seven or more, eight or more, nine or more, or ten or more distinct detectable markers having emission lifetimes that are distinguishable from each other.

In certain aspects, distinct detectable markers can have one or more optical properties (e.g., emission spectra, emission intensities, emission wavelengths, emission lifetimes, etc.) that are independently or semi-independently controllable. In some aspects, the encoded detectable markers can include two or more, three or more, four or more, five or more, six or more, seven or more, eight or more, nine or more, or ten or more distinct detectable markers having emission spectra that are independently or semi-independently controllable. In some aspects, the encoded detectable marks can include two or more, three or more, four or more, five or more, six or more, seven or more, eight or more, nine or more, or ten or more distinct detectable markers having emission intensities that are independently or semi-independently controllable. In some aspects, the encoded detectable marks can include two or more, three or more, four or more, five or more, six or more, seven or more, eight or more, nine or more, or ten or more distinct detectable markers having emission wavelengths that are independently or semi-independently controllable. In some aspects, the encoded detectable marks can include two or more, three or more, four or more, five or more, six or more, seven or more, eight or more, nine or more, or ten or more distinct detectable markers having emission lifetimes that are independently or semi-independently controllable.

In some aspects of the present disclosure, an apparatus for performing a multiplexed screening comprises: a microtiter plate; a first biological cell comprising a first detectable marker and a first genotype; a second biological cell comprising a second detectable marker and a second genotype, wherein the second genotype comprises a mutation relative to the first genotype; a compound; a first detection apparatus configured to detect the first detectable marker; and a second detection apparatus configured to detect the second detectable marker. The first detection apparatus can detect the detectable marker code of the first biological cell. The second detection apparatus can detect the detectable marker code of the second biological cell.

Data sets collected as part of the methods described herein can be analyzed individually or in comparison with each other. Data resulting from the methods and/or apparatus disclosed herein can also be analyzed in real-time (individually or with consideration of other collected data), stored for subsequent integration with other data sets, or either stored or output for manual analysis or analysis using a separate data analysis protocol. These data can also be used to determine efficacy of a given candidate compound with respect to effects on several aspects of the cultured cells, including cell viability, function (e.g., proliferation, migration, differentiation), or phenotype (e.g., morphology). Collected data and the quantitative or qualitative derivations thereof can further be used to determine cellular mechanisms, the progression and/or treatment of abnormal conditions represented by the cells comprising the invention, strategies for drug development, and the selection of therapeutic agents in a clinical or preclinical setting.

In the course of experimentation, analysis of data can lead to identification of pathways, individual compounds, and combinations of treatments amenable to treating cells with a given abnormal condition. These compounds or the set of compounds shown to modulate these pathways can comprise a library of compounds that could potentially prove efficacious in subsequent in vivo or ex vivo treatment strategies, where the term “efficacious” is used here to describe a treatment that improves clinical or cosmetic endpoints of biological organisms or tissues intended for use in biological applications. As such, the libraries of candidate compounds identified using this invention can be subjected to further testing to validate efficacy in treating the relevant condition, including ligand specificity (e.g., Cellzome), mechanism of action testing, molecular modeling. For example, small molecules and other drugs can be subjected to NMR analysis in which Z-score, hydrophobicity, and molecular weight are measured and considered in view of accepted guidelines for drug development, such as the Lipinski's “rule of five” or the related “rule of three”.

The methods of this invention can include real-time analysis of recorded data or storage of data for subsequent analysis. In either case, data collected for each individual metric can be evaluated alone or in conjunction with data collected from other metrics. For example, the transcription of a fluorescently-tagged protein in a subset of cells can be analyzed in light of morphological changes in the same or other cells with respect to the time point at which each event occurred.

### Data Processing and Digital Processing Device

The systems, apparatuses, and methods described herein can include a digital processing device, or use of the same. The digital processing device can include one or more hardware central processing units (CPU) that carry out the device's functions. The digital processing device can further comprise an operating system configured to perform executable instructions. In some instances, the digital processing device is optionally connected to a computer network, is optionally connected to the Internet such that it accesses the World Wide Web, or is optionally connected to a cloud computing infrastructure. In other instances, the digital processing device is optionally connected to an intranet. In other instances, the digital processing device is optionally connected to a data storage device.

In accordance with the description herein, suitable digital processing devices can include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers can include those with booklet, slate, and convertible configurations, known to those of skill in the art.

The digital processing device can include an operating system configured to perform executable instructions. The operating system can be, for example, software, including programs and data, which can manage the device's hardware and provides services for execution of applications. Those of skill in the art will recognize that suitable server operating systems can include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some cases, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some instances, the device can include a storage and/or memory device. The storage and/or memory device can be one or more physical apparatuses used to store data or programs on a temporary or permanent basis. In some instances, the device is volatile memory and requires power to maintain stored information. In other instances, the device is non-volatile memory and retains stored information when the digital processing device is not powered. In still other instances, the non-volatile memory comprises flash memory. The non-volatile memory can comprise dynamic random-access memory (DRAM). The non-volatile memory can comprise ferroelectric random access memory (FRAM). The non-volatile memory can comprise phase-change random access memory (PRAM). The device can be a storage device including, by way of non-limiting examples, CD-ROMs, DVDs, flash memory devices, magnetic disk drives, magnetic tapes drives, optical disk drives, and cloud computing based storage. The storage and/or memory device can also be a combination of devices such as those disclosed herein.

The digital processing device can include a display to send visual information to a user. The display can be a cathode ray tube (CRT). The display can be a liquid crystal display (LCD). Alternatively, the display can be a thin film transistor liquid crystal display (TFT-LCD). The display can further be an organic light emitting diode (OLED) display. In various cases, on OLED display is a passive-matrix OLED (PMOLED) or active-matrix OLED (AMOLED) display. The display can be a plasma display. The display can be a video projector. The display can be a combination of devices such as those disclosed herein.

The digital processing device can also include an input device to receive information from a user. For example, the input device can be a keyboard. The input device can be a pointing device including, by way of non-limiting examples, a mouse, trackball, track pad, joystick, game controller, or stylus. The input device can be a touch screen or a multi-touch screen. The input device can be a microphone to capture voice or other sound input. The input device can be a video camera or other sensor to capture motion or visual input. Alternatively, the input device can be a Kinect™, Leap Motion™, or the like. In further aspects, the input device can be a combination of devices such as those disclosed herein.

### Non-Transitory Computer Readable Storage Medium

In some instances, the systems, apparatus, and methods disclosed herein can include one or more non-transitory computer readable storage media encoded with a program including instructions executable by the operating system of an optionally networked digital processing device. In further instances, a computer readable storage medium is a tangible component of a digital processing device. In still further instances, a computer readable storage medium is optionally removable from a digital processing device. A computer readable storage medium can include, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media. Computer program

The systems, apparatus, and methods disclosed herein can include at least one computer program, or use of the same. A computer program includes a sequence of instructions, executable in the digital processing device's CPU, written to perform a specified task. In some embodiments, computer readable instructions are implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain embodiments, is written in various versions of various languages.

The functionality of the computer readable instructions can be combined or distributed as desired in various environments. A computer program can comprise one sequence of instructions. A computer program can comprise a plurality of sequences of instructions. In some instances, a computer program is provided from one location. In other instances, a computer program is provided from a plurality of locations. In additional cases, a computer program includes one or more software modules. Sometimes, a computer program can include, in part or in whole, one or more web applications, one or more mobile applications, one or more standalone applications, one or more web browser plug-ins, extensions, add-ins, or add-ons, or combinations thereof.

### Web Application

A computer program can include a web application. In light of the disclosure provided herein, those of skill in the art will recognize that a web application, in various aspects, utilizes one or more software frameworks and one or more database systems. In some cases, a web application is created upon a software framework such as Microsoft® .NET or Ruby on Rails (RoR). In some cases, a web application utilizes one or more database systems including, by way of non-limiting examples, relational, non-relational, object oriented, associative, and XML database systems. Sometimes, suitable relational database systems can include, by way of non-limiting examples, Microsoft® SQL Server, mySQL™, and Oracle®. Those of skill in the art will also recognize that a web application, in various instances, is written in one or more versions of one or more languages. A web application can be written in one or more markup languages, presentation definition languages, client-side scripting languages, server-side coding languages, database query languages, or combinations thereof. A web application can be written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). A web application can be written to some extent in a client-side scripting language such as Asynchronous Javascript and XML (AJAX), Flash® Actionscript, Javascript, or Silverlight®. A web application can be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. Sometimes, a web application can be written to some extent in a database query language such as Structured Query Language (SQL). Other times, a web application can integrate enterprise server products such as IBM® Lotus Domino®. In some instances, a web application includes a media player element. In various further instances, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

A computer program can include a mobile application provided to a mobile digital processing device. In some cases, the mobile application is provided to a mobile digital processing device at the time it is manufactured. In other cases, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

A computer program can include a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. A computer program can include one or more executable complied applications.

### Web Browser Plug-In

The computer program can include a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) can be software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The systems and methods disclosed herein can include software, server, and/or database modules, or use of the same. In view of the disclosure provided herein, software modules can be created by techniques known to those of skill in the art using machines, software, and languages known to the art. The software modules disclosed herein can be implemented in a multitude of ways. A software module can comprise a file, a section of code, a programming object, a programming structure, or combinations thereof. A software module can comprise a plurality of files, a plurality of sections of code, a plurality of programming objects, a plurality of programming structures, or combinations thereof. In various aspects, the one or more software modules comprise, by way of non-limiting examples, a web application, a mobile application, and a standalone application. In some instances, software modules are in one computer program or application. In other instances, software modules are in more than one computer program or application. In some cases, software modules are hosted on one machine. In other cases, software modules are hosted on more than one machine. Sometimes, software modules can be hosted on cloud computing platforms. Other times, software modules can be hosted on one or more machines in one location. In additional cases, software modules are hosted on one or more machines in more than one location.

### Databases

The methods, apparatus, and systems disclosed herein can include one or more databases, or use of the same. In view of the disclosure provided herein, those of skill in the art will recognize that many databases are suitable for storage and retrieval of analytical information described elsewhere herein. In various aspects described herein, suitable databases can include, by way of non-limiting examples, relational databases, non-relational databases, object oriented databases, object databases, entity-relationship model databases, associative databases, and XML databases. A database can be internet-based. A database can be web-based. A database can be cloud computing-based. Alternatively, a database can be based on one or more local computer storage devices.

### Kits

The invention can also comprise a kit of components used for the resolution of heterogeneous cell populations in a single vessel. The kit can include a microtiter plate (e.g., uncoated microtiter plates or pre-coated microtiter plates), a plasmid encoding a TALEN backbone, and instructions for performing the methods described herein. The kit can further include an aliquot of K562 leukemic cells (or a genetic variant thereof), an aliquot of antibiotics (e.g., ampicillin, chloramphenicol, kanamycin, tetracycline, doxycycline, spectinomycin, coumermycin, carbenicillin, bleocin, or gentamycin), culture medium, a plasmid encoding a detectable marker, plasmids containing repeat variable diresidues (RVDs), aliquots of nucleotides (either in the form of individual aliquots or as an aliquot of mixed nucleotides), or an aliquot of a cell line relevant to an abnormal condition (e.g., LXFL 529, DMS 114, SHP-77, DLD-1, KM20L2, SNB-78, XF498, RPMI-7951, M19-MEL, RXF-631, SN12K1, P388, P338/ADR, and the NCI 60 cell line list, which comprises the lines CCRF-CEM, HL-60, K562, MOLT-4, RPMI-8226, SR leukemic cells, A549, EKVX, HOP-62, HOP-92, H226, H23, H332M, H460, H522, COLO 205, HCC-2998, HCT-116, HCT-15, HT29, KM12, SW-620, SF-268, SF-295, SF-539, SNB-19, SNB-75, U251, LOX IMVI, MALME-3M, M14, MDA-MB-435, SK-MEL-2, SK-MEL-28, SK-MEL-5, UACC-257, UACC-62, IGR-OV1, OVCAR-3, OVCAR-4, OVCAR-5, OVCAR-8, ADR-RES, SK-OV-3, 786-O, A498, ACHN, CAM-1, RXF 393, SN12C, TK-10, UO-31, PC-3, DU-145, MCF7, MDA-MB-231, MDA-MB-468, HS578T, MDA-N, BT-549, and T-47D).

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

### EXAMPLES

The following examples are included to further describe some aspects of the present disclosure, and should not be used to limit the scope of the invention.

### Example 1

### Engineering a DNA Mutation Using TALENs

This example describes the use of transcription activator-like effector nucleases (TALENs) to engineer a genetic variation or mutation of interest into a cell. The DNA binding domain of a TALEN is selected for its ability to bind to a specific DNA sequence in which the mutation will be introduced. This TALEN is inserted into a vector, which is then transfected into the cell of interest. Alternatively, the TALEN is delivered as mRNA (which is then translated into protein) or directly as functional protein. The donor DNA, which includes two homology arms identical to a portion of the targeted genomic region and a DNA segment between the homology arms that encodes the mutation/alteration of interest, is also introduced. The DNA of the cell is cut by the TALEN at the specific, targeted genomic location and the mutation of interest (carried on the donor DNA) is inserted into that genomic locus by homologous recombination. Thus, the cell with the mutation/alteration of interest at the endogenous locus is produced. Cells created in this manner are screened by polymerase chain reaction (PCR) and sequencing to confirm successful genomic editing.

### Example 2

### Engineering a DNMT3A Mutation into K562 Leukemic Cells

This example describes the use of transcription activator-like effector nucleases (TALENs) to engineer a heterozygous p.R882H DNMT3A mutation into the K562 leukemic cell line. The DNA binding domains of two TALEN pairs are selected for their abilities to bind to specific DNA sequences on either side of the sequence of the endogenous DNMT3A gene where the p.R882H mutation is to be introduced. A DNA delivery vector encoding the TALEN pairs specific to the targeted loci of the DNMT3A gene is introduced into K562 leukemic cells along with donor (e.g., template) DNA, which encodes the p.R882H mutation of the DNMT3A. The TALEN pairs are allowed to cut the endogenous DNMT3A sequence of the K562 leukemic cells at the prescribed loci, and the cell's machinery is allowed to incorporate the donor (e.g., template) DNA that encodes the p.R882H mutation into the endogenous genomic locus through homologous recombination. K562 leukemic cells that are heterogeneous for the p.R882H mutation are distinguished from K562 leukemic cell that are homogeneous for the p.R882H by single cell cloning and sequencing. Briefly, edited K562 leukemic cells are cloned by dilution and then sorted based on expression of a detectable marker before being screened for copy number of the introduced mutation by sequencing.

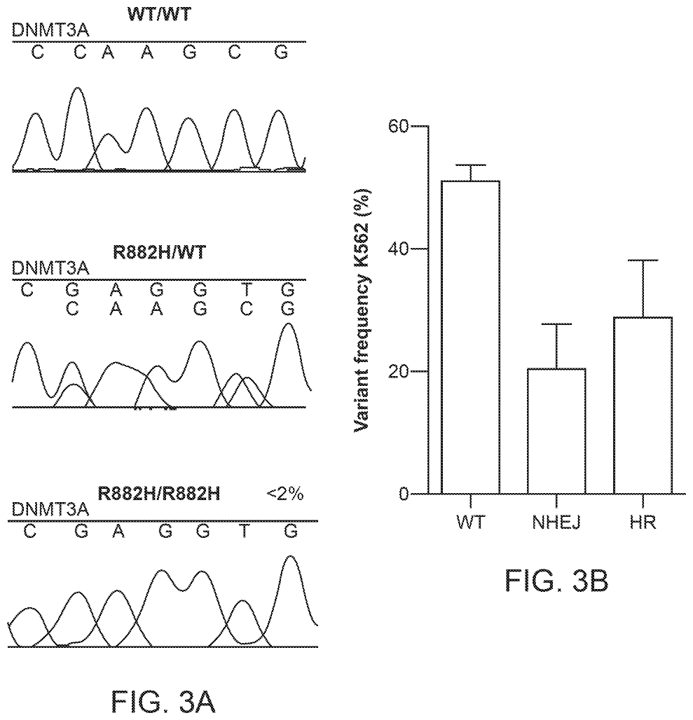

FIG. 3 shows variant frequency of R882H mutations introduced into K562 cells by TALENs. FIG. 3A shows Sanger sequencing of TALEN-edited K562 single clones in which the DNMT3A mutation was integrated into the cells (WT/WT), in which one copy of DNMT3A mutation (R882H/WT) was integrated into the cells, and in which two copies of DNMT3A mutation (R882H/R882H) was incorporated into the cells. FIG. 3B shows the variant frequency of TALEN-edited K562 cells, in which WT indicated the no integration of the DNMT3A mutation, NHEJ indicates integration of the DNMT3A mutation by non-homologous end joining, and HR indicates integration of the DNMT3A mutation by homologous recombination.

### Example 3

### Fluorescent Labeling of Cells

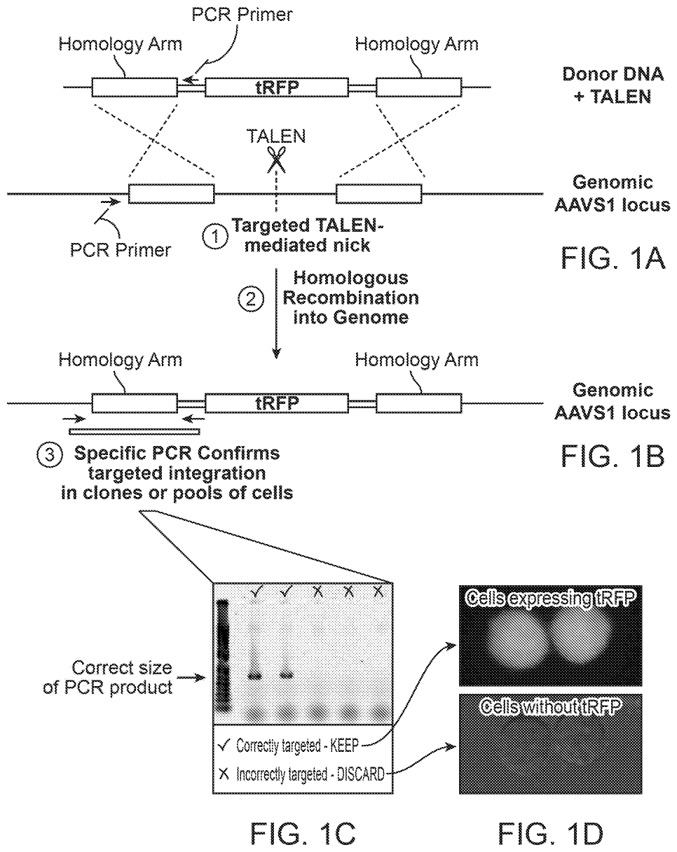

This example describes the fluorescent labeling of cells. Cells are transduced with a TALEN that cuts within a designated region of the endogenous AAVS1 safe harbor locus and a donor DNA containing a fluorescent protein expression cassette. The fluorescent protein is stably expressed, and clones expressing the fluorescent protein are isolated by flow sorting or serial dilution methods. The insertion locus of the fluorescent protein cassette corresponds to a position in which it is expressed under the control of endogenous cellular machinery that correspond to a relevant protein or cellular signaling pathway (e.g., a housekeeping gene, a protein related to the cell cycle, or a protein of interest in the cellular pathway being studied). A selectable marker (e.g., puromycin resistance) is included in the donor DNA that is integrated by homologous recombination to enable drug-mediated killing of non-stably integrated cells. Rapid assessment of successful integration of the fluorescent protein cassette is performed by PCR using primers that flank the integration site in the genome and the donor DNA (as in FIG. 1A). The donor DNA is successfully and specifically inserted if the amplification is successful. If additional PCR primer pairs are deemed necessary for validation of donor DNA insertion, additional primer pairs are designed to recognize sequences on either side of the genomic locus chosen for insertion. PCR products from successful integration will be larger than PCR products from unsuccessful integration by exactly the length of the inserted donor DNA, which is determined by DNA gel analysis. Alternatively, fluorescence microscopy is used to evaluate success and permanence of fluorescent marker integration. Sometimes, this cell labeling strategy is employed serially with another TALEN-mediated engineering assay that is directed toward a separate, biologically relevant locus of interest, as described in EXAMPLE 1, or multiple TALEN-mediated engineering events are performed at the same time on the same cells.

### Example 4

### Fluorescence Labeling of K562 Leukemic Cells

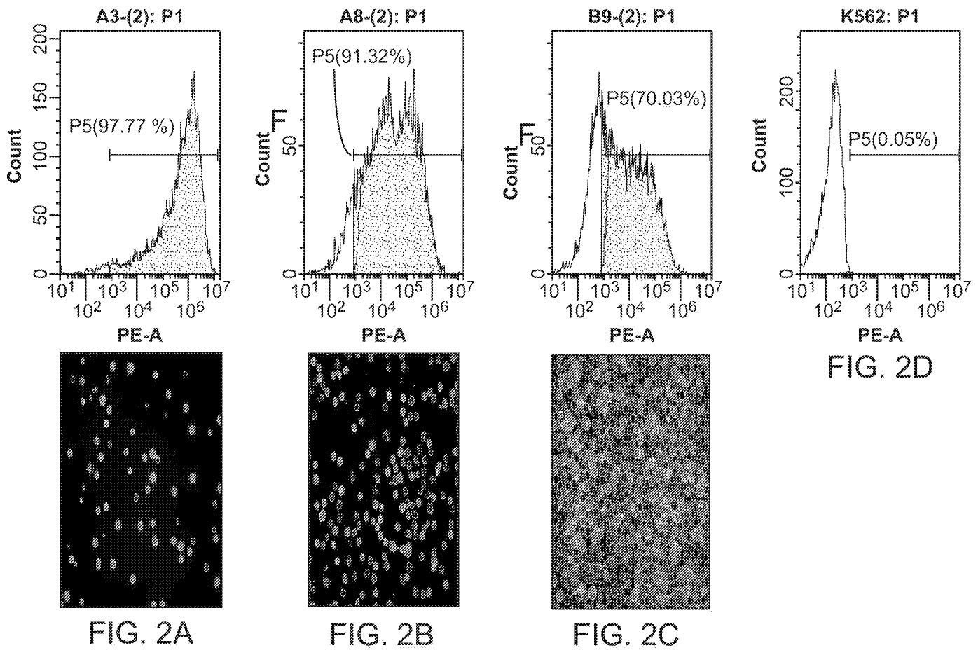

This example describes how K562 leukemic cells are fluorescently labeled with red fluorescent protein (RFP). K562 leukemic cells were transfected with both donor DNA and DNA encoding a TALEN pair that cuts within a designated region of a selected AAVS1 safe harbor site for fluorescent marker DNA insertion cassette. The donor DNA encoded RFP, the sequence of which was flanked by nucleic acid arms homologous to regions on either side of the safe harbor insertion site. Three clones were tested for successful insertion of the fluorescent marker cassette, which was verified by flow cytometry and fluorescent microscopy. FIG. 2 shows the fluorescence of three K562 clones after transcription activator-like effector nuclease (TALEN)-mediated cell labeling with a fluorescent marker. FIG. 2A shows the stable expression of RFP fluorescence by cells of clone A3 after targeted AAVS1 integration as shown by flow cytometry (top) and microscopy (bottom). FIG. 2B shows the stable expression of RFP fluorescence by cells of clone A8 after targeted AAVS1 integration as shown by flow cytometry (top) and microscopy (bottom). FIG. 2C shows the stable expression of RFP fluorescence by cells of clone B9 after non-targeted AAVS1 integration as shown by flow cytometry (top) and microscopy (bottom). FIG. 2D shows no fluorescence by K562 cells without AAVS1 integration by flow cytometry.

### Example 5

### Fluorescence Labeling of K562 Leukemic Cells and p.R882H Mutant K562 Leukemic Cells

This example describes how K562 leukemic cells and p.R882H mutant K562 leukemic cells are fluorescently labeled. K562 leukemic cells are transfected with both donor DNA and DNA encoding a TALEN pair that cuts within a designated region of a selected AAVS1 safe harbor site for fluorescent marker DNA insertion cassette. The donor DNA encodes mCherry, the sequence of which is flanked by nucleic acid arms homologous to regions on either side of the safe harbor insertion site. The safe harbor insertion site is selected based on its ability to achieve expression levels of the inserted fluorescent marker cassette substantially similar to endogenous expression of DNMT3A. Successful insertion of the fluorescent marker cassette is verified by flow cytometry followed by PCR verification and/or flow cytometry. p.R882H mutant K562 leukemic cells are created in a similar manner as described above, with the exceptions that the donor DNA and TALEN DNA are transfected into a cell line known to harbor the p.R882H mutation in the DNMT3A gene. Clones with successful insertion of mCherry sequences are screened by flow cytometry and verified by Sanger sequencing and PCR analysis.

### Example 6

### Isotopic Labeling of Cells

This example describes how wild-type cells and TALEN-edited cells are isotopically labeled. The TALEN-edited cells in this example are produced by the method described in EXAMPLE 1. Wild-type cells are cultured in a medium with amino acids containing a first heavy isotope. Wild-type cells with the first heavy isotope are produced. TALEN-edited cells are separately cultured in a medium with amino acids containing a second heavy isotope. TALEN-edited cells with the second heavy isotope are produced.

### Example 7

### Organelle Labeling of Cells

This example describes how organelles in wild-type cells and TALEN-edited cells are labeled. The TALEN-edited cells in this example are produced by the method described in EXAMPLE 2. Briefly, TALEN DNA encoding a TALEN pair specific for a safe harbor insertion site and donor DNA containing a pair of homology arms directed toward the regions flanking the selected safe harbor insertion site and an expression cassette are transfected into a cell. The expression cassette encodes an mCherry-actin fusion protein, and successful insertion is verified by flow cytometry, PCR analysis, and/or sequencing of the insertion region. Thus, wild-type cells labeled with the fluorescent tag in the first organelle are produced. A second plasmid containing a eGFP-mitofusin-1 fusion protein with a sequence that directs eGFP-mitofusin-1 fusion protein to a label second organelle is transfected into positive clones from the first gene editing event. Positive clones from the second labeling event are screened and verified, thus producing TALEN-edited cells labeled with two labeled proteins.

Alternatively, a first plasmid containing mCherry-actin fusion protein with a sequence that directs the mCherry-actin fusion protein to label an organelle is transfected into wild-type cells. Thus, wild-type cells which are labeled with mCherry-actin fusion protein in that organelle are produced. A second plasmid containing an eGFP-mitofusin-1 fusion protein with a sequence that directs the eGFP-mitofusin-1 fusion protein to a label an organelle (in which the same organelle is labeled as is labeled in the wild-type cells) is transfected into the TALEN-edited cells. Thus, TALEN-edited cells labeled with the eGFP-mitofusin-1 fusion protein in that organelle are produced.

### Example 8

### Detection of Different Cell Populations with the Same Fluorescent Marker

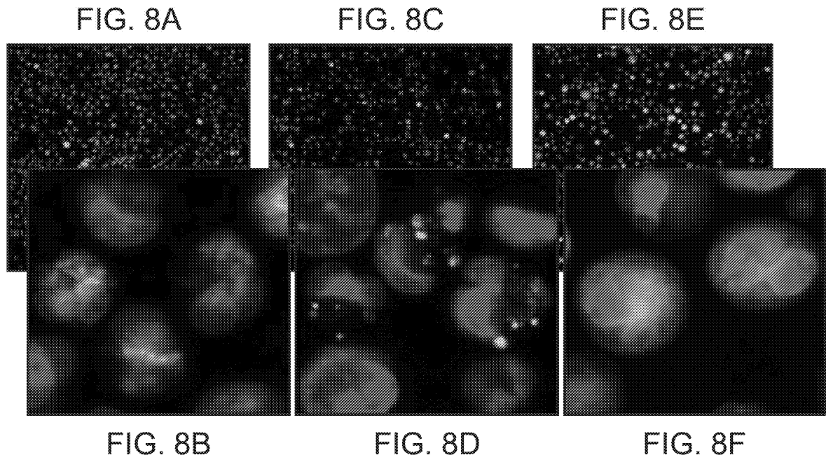

This example describes how different cell populations can be detected when labeled with the same fluorescent marker. Separate populations of live 786-O cells were labeled with either a lysosome dye tracker, mitochondria dye tracker, or an endoplasmic reticulum dye tracker. FIG. 8 shows three different cell populations identified by organelle tags using the same fluorescent marker. FIG. 8A shows 786-O cells labeled by an organelle tracker dye that localizes to mitochondria. FIG. 8B shows a higher magnification of FIG. 8A, showing the specific pattern of dye localization to the mitochondria of a cell. FIG. 8C shows 786-O cells labeled by an organelle tracker dye that localizes to lysosomes. FIG. 8D shows a higher magnification of FIG. 8C, showing the specific pattern of dye localization to the lysosomes of a cell. FIG. 8E shows 786-O cells labeled by an organelle tracker dye that localizes to endoplasmic reticulum. FIG. 8F shows a higher magnification of FIG. 8C, showing the specific pattern of dye localization to the endoplasmic reticulum of a cell.

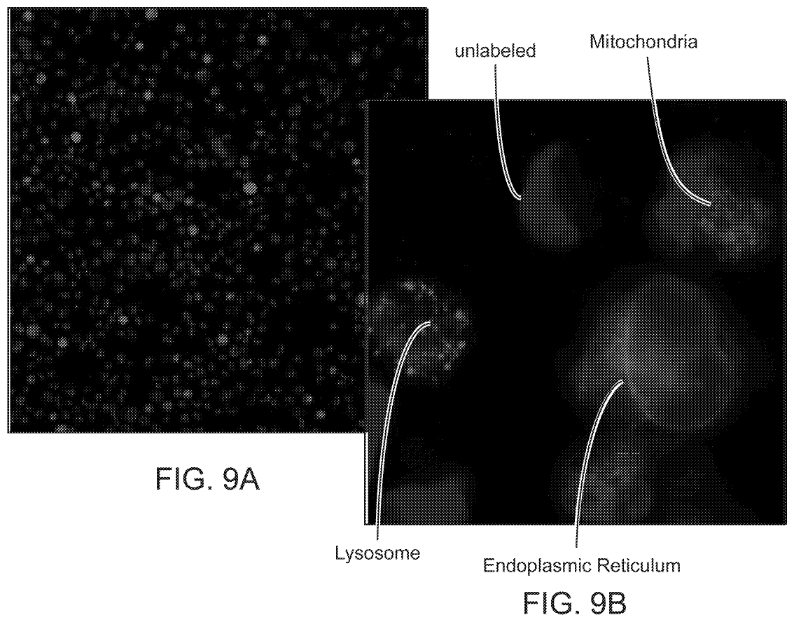

These separate populations were then mixed with a control population of cells that was unlabeled. Fluorescence microscopy was used to detect the organelle dye trackers in the mixed population of cells. FIG. 9A shows a mixed population of live 786-O cells that were separately labeled with specific organelle tracker dyes. FIG. 9B shows a higher magnification H-image of the mixed population of live 786-O cells with the same fluorescent marker from FIG. 9A, but in which the populations are distinguished by the localization of fluorescent marker to either the mitochondria, lysosomes, or the endoplasmic reticulum. Unlabeled cells were used as a negative control.

### Example 9

### Multiplexed High-Throughput Screening

This example describes how a multiplexed high-throughput screen is performed. Wild-type cells labeled by any of the methods described in EXAMPLE 3, EXAMPLE 5, or EXAMPLE 6 are mixed with TALEN-edited cells labeled by any of the methods described in EXAMPLE 3, EXAMPLE 5, or EXAMPLE 6 and added to multi-titer plates, the wells of which have been pre-coated with dried compound such that when an equal volume of culture medium is added to each well, final compound concentrations are achieved in each well. Individual cell lines are mixed and delivered to the well in the appropriate volume of culture medium by robotic dispensation methods; however, microfluidic or ultrasonic dispensation methods may be used if robotic dispensation methods are not available or practical. After a specific period of time, the cell counts and ratios for each cell line, viability of each cell line, and metabolic profile of each cell line are assessed at each designated time point. Additional reagents, such as additional compound or differentiation factors, are added to the wells at the designated time points by the same dispensation method as cells were added to the wells. Cells are fixed or detached from culture and subjected to further analysis of viability, proliferation, and metabolic profile, by flow cytometry and mass spectrometry. Throughout experimentation, control cells present in the same well as mutant cell(s) of interest are evaluated to identify and eliminate non-specific and null effects on cells of interest.

### Example 10

### Multiplexed High-Throughput Screen for a Therapeutic Compound for p.R882H DNMT3 Mutation

This example describes how a multiplexed high-throughput screen for compounds that inhibit the p.R882H mutation is performed. K562 leukemic cells labeled with eGFP are mixed together with heterozygous p.R882H mutant K562 leukemic cells labeled with mCherry in an appropriate (pre-determined) ratio. An initial library of approximately 10,000 compounds representing the large diversity of chemical compound space is used for screening with the understanding that this may be expanded to approximately 200,000 compounds with greater diversity or further expanded to a full compound collection containing as many as 2-3 million compounds or more, based on experimental results. Initially, a library of 10,000 compounds with the potential to affect the p.R882H mutation cells is selected. Each compound from this library is separately added to a well. After a specified period of time, each well is screened for the eGFP K562 leukemic cell label and the mCherry p.R882H mutant K562 leukemic cell label. Flow cytometry or microscopy is used to detect the labels, and the type of labeling (e.g., presence/absence of detectable marker signal, detectable marker pattern, signal intensity, localization of detectable marker with respect to organelles, cellular structures, and other detectable markers) are evaluated. The type of eGFP K562 leukemic cell labeling detected in analyzed cells is compared to the type of fluorescent mCherry p.R882H mutant K562 leukemic cell labeling detected in analyzed cells. If a greater number of K562 leukemic cells are detected, as identified by their fluorescent eGFP label, than the number of detected p.R882H mutant K562 leukemic cells, as identified by their mCherry label, the compound that was added to that well is chosen for further testing as a potential candidate that can be used as a therapeutic against cells harboring the p.R882H mutation.

This example describes another method for how a multiplexed high-throughput screen for compounds that inhibit the p.R882H mutation is performed. K562 leukemic cells are labeled by eGFP. Heterozygous p.R882H mutant K562 leukemic cells are labeled by YFP. Homozygous p.R882H mutant K562 leukemic cells are labeled by mCherry. Heterozygous p.R882H/NPM1 double mutant K562 leukemic cells are produced by the method in EXAMPLE 1 to engineer a NPM1 mutation into heterozygous p.R882H mutant K562 leukemic cells. Heterozygous p.R882H/NPM1 double mutant K562 leukemic cells are labeled by mCerulean. Homozygous p.R882H/NPM1 double mutant K562 leukemic cells are produced by the method in EXAMPLE 1 to engineer a NPM1 mutation into homozygous p.R882H mutant K562 leukemic cells. Homozygous p.R882H/NPM1 double mutant K562 leukemic cells are labeled by mPlum. A library of compounds with the potential to affect the p.R882H mutant cells is then selected. Each compound from this library is separately added to a well. After a specified period of time, each well is screened for the presence of each cell-type label. Flow cytometry or microscopy is used to detect the label, depending on whether the time point is terminal or intermediate in the experimental protocol. The level of detected labels for each cell type is used to determine the compounds for further testing as potential candidates that can be used as a therapeutic against the p.R882H mutation.

### Example 11

### Multiplexed High-Throughput Screen for a Therapeutic Compound for p.R882H DNMT3 Mutation Using Detectable Marker Pattern

This example describes how a multiplexed high-throughput screen for compounds that inhibit the p.R882H mutation is performed using detectable marker patterns. K562 leukemic cells labeled with eGFP that localizes to mitochondria are mixed together in an appropriate (pre-determined) ratio with heterozygous p.R882H mutant K562 leukemic cells labeled with eGFP that localizes to lysosomes. An initial library of approximately 10,000 compounds representing the large diversity of chemical compound space is used for screening with the understanding that this may be expanded to approximately 200,000 compounds with greater diversity or further expanded to a full compound collection containing as many as 2-3 million compounds or more, based on experimental results. Initially, a library of 10,000 compounds with the potential to affect the p.R882H mutation cells is selected. Each compound from this library is separately added to a well. After a specified period of time, each well is screened for the eGFP localization pattern, in which K562 leukemic cells are identified by eGFP localization to the mitochondria and p.R882H mutant K562 leukemic cells are identified by localization to lysosomes. Microscopy is used to detect the detectable marker pattern associated with organelle localization, which is evaluated by manual image analysis or computer-assisted image analysis. If a greater number of K562 leukemic cells are detected, as identified by their fluorescent eGFP label localization to mitochondria, than the number of detected p.R882H mutant K562 leukemic cells, as identified by their eGFP label localization to lysosomes, the compound that was added to that well is chosen for further testing as a potential candidate that can be used as a therapeutic against cells harboring the p.R882H mutation. By distinguishing different cell types based on localization patterning using a single color fluorescent, other color channels are opened for subsequent multiparametric biological readout. For example, a fluorescently labeled Lamin A antibody or CellLight fluorescent nucleus probes for assaying nuclear integrity, CellMask plasma membrane stain for assaying cell plasma membrane, or RedoxSensor Red CC-1 for assaying the oxidative state of the cell cytoplasm is used for further analyzing the effect of different compounds on the K562 leukemic cells versus the p.R882H mutant K562 leukemic cells.

This example describes another method for how a multiplexed high-throughput screen for compounds that inhibit the p.R882H mutation is performed using detectable marker pattern. K562 leukemic cells are labeled by eGFP that localizes to mitochondria. Heterozygous p.R882H mutant K562 leukemic cells are labeled by eGFP that localizes to lysosomes. Heterozygous p.R882H/NPM1 double mutant K562 leukemic cells are produced by the method in EXAMPLE 1 to engineer a NPM1 mutation into heterozygous p.R882H mutant K562 leukemic cells. Heterozygous p.R882H/NPM1 double mutant K562 leukemic cells are labeled by eGFP that localizes to endoplasmic reticulum. A library of compounds with the potential to affect the p.R882H mutant cells is then selected. Each compound from this library is separately added to a well. After a specified period of time, each well is screened for the presence of each cell-type label pattern of localization. Microscopy is used to detect this detectable marker pattern associated with organelle localization, which is evaluated by manual image analysis or computer-assisted image analysis. The number of each cell type, as identified by their detectable marker localization pattern, is used to determine the compounds for further testing as potential candidates that can be used as a therapeutic against the p.R882H mutation and p.R882H/NPM1 double mutation. By distinguishing different cell types based on localization patterning using a single color fluorescent, other color channels are opened for subsequent multiparametric biological readout. For example, a fluorescently labeled Lamin A antibody or CellLight fluorescent nucleus probes for assaying nuclear integrity, CellMask plasma membrane stain for assaying cell plasma membrane, or RedoxSensor Red CC-1 for assaying the oxidative state of the cell cytoplasm is used for further analyzing the effect of different compounds on each cell type.

### Example 12

### Cytotoxicity Assay Using a Multiplexed High-Throughput Screen

This example describes how a cytotoxicity assay using a multiplexed high-throughput screen is performed. K562 cells labeled by any of the methods described in EXAMPLE 3, EXAMPLE 5, or EXAMPLE 6 are mixed with TALEN-edited cells labeled by any of the methods described in EXAMPLE 3, EXAMPLE 5, or EXAMPLE 6 and added to multi-titer plates, the wells of which have been pre-coated with dried compound such that when an equal volume of culture medium is added to each well, final compound concentrations are achieved in each well. Each population of cells are mixed in equal numbers and added to the well in specific volume of culture medium by robotic dispensation methods. After a specific period of time, the cell counts and ratios for each cell population is assessed to determine the cytotoxicity of the compound that is being tested. The cell number of the labeled K562 cells that do not harbor a p.R882H mutation is found to be greater than the number of TALEN-edited cells harboring a p.R882H mutation, indicating that the compound has specific cytotoxicity for the TALEN-edited cells.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of multiplexed screening, the method comprising:
a) providing a plurality of vessels, wherein each vessel comprises:
i) a first biological cell comprising a first detectable marker and a first genotype; and
ii) a second biological cell comprising a second detectable marker and a second genotype, wherein the second genotype comprises a genetic variation relative to the first genotype;
b) contacting the first biological cell and the second biological cell with a compound in each vessel; and
c) detecting the first detectable marker and the second detectable marker after the contacting in each vessel.

2. The method of claim 1, further comprising quantifying the level of the first detectable marker and the second detectable marker in each vessel.

3. The method of claim 1, wherein the first detectable marker is a fluorescent marker or an isotopic label or the second detectable marker is a fluorescent marker or an isotopic label.

4. The method of claim 1, wherein the first detectable marker labels a membrane or organelle of the first biological cell or the second detectable marker labels a membrane or organelle of the second biological cell.

5. (canceled)

6. (canceled)

7. The method of claim 1, wherein the first biological cell or the second biological cell comprise more than one detectable marker.

8. The method of claim 7, wherein the more than one detectable marker is a fluorescent marker or an isotopic label.

9. (canceled)

10. The method of claim 1, further comprising analyzing the first biological cell or second biological cell using flow cytometry.

11. The method of claim 1, wherein the detecting is by one or more of mass spectrometry, optical detection, and microscopy.

12. (canceled)

13. (canceled)

14. The method of claim 1, wherein the first biological cell and the second biological cell are from a subject.

15. The method of claim 1, wherein the genetic variation in the second genotype is engineered by a gene editing tool comprising a transcription activator-like effector nuclease (TALEN), a zinc finger nuclease, or CRISPR/Cas9 or wherein the genetic variation is a heterozygous or a homozygous genetic variation associated with a disease.

16.-23. (canceled)

24. The method of claim 1, wherein the compound is a drug and the method comprises determining the effect of the drug on the first biological cell and the second biological cell.

25. (canceled)

26. An apparatus for multiplexed screening, the apparatus comprising:
a) a microtiter plate;
b) a first biological cell comprising a first detectable marker and a first genotype; c) a second biological cell comprising a second detectable marker and a second genotype, wherein the second genotype comprises a genetic variation relative to the first genotype;
d) a compound;
e) a first detection apparatus configured to detect the first detectable marker; and f) a second detection apparatus configured to detect the second detectable marker.

27. The apparatus of claim 26, wherein the first detectable marker is a fluorescent marker or an isotopic label or the second detectable marker is a fluorescent marker or an isotopic label.

28. The apparatus of claim 26, wherein the first detectable marker labels a membrane or organelle of the first biological cell or the second detectable marker labels a membrane or organelle of the second biological cell.

29. (canceled)

30. (canceled)

31. The apparatus of claim 26, wherein the first biological cell or the second biological cell comprises more than one detectable marker.

32. The apparatus of claim 31, wherein the more than one detectable marker is a fluorescent marker or an isotopic label.

33. (canceled)

34. The apparatus of claim 26, wherein the first detection apparatus is the same as the second detection apparatus.

35. The apparatus of claim 26, further comprising a flow cytometer or a microscope.

36. The apparatus of claim 26, wherein the first detection apparatus or the second detection apparatus comprises a mass spectrometer or the first detection apparatus or the second detection apparatus comprises an optical detector.

37. (canceled)

38. (canceled)

39. The apparatus of claim 26, wherein the first biological cell and the second biological cell are from a subject and wherein the genetic variation in the second genotype is engineered by a gene editing tool comprising a transcription activator-like effector nuclease (TALEN), a zinc finger nuclease, or CRISPR/Cas9 or wherein the genetic variation is a heterozygous or a homozygous genetic variation associated with a disease.

40.-55. (canceled)
